(19) United States
(12) Patent Application Publication
(10) Pub. No.: US 2002/0137022 A1
(43) Pub. Date: Sep. 26, 2002

(54) Methods and compositions for the construction and use of envelope viruses as display particles

(76) Inventor: Min Li, Lutherville, MD (US)

Correspondence Address: Robin M. Silva, FLEHR HOHBACH TEST, ALBRITTON & HERBERT LLP, Four Embarcadero Center-Suite 3400, San Francisco, CA, 94111-4187, US

(21) Appl. No.: 09/922,503

(22) Filed: Aug. 2, 2001

Related U.S. Application Data
(60) Provisional application No. 60/222,697, filed on Aug. 2, 2000.

(51) Int. Cl. C12Q 1/70; C12Q 1/68; G01N 33/53; C07H 21/04

(52) U.S. Cl. 435/5; 435/6; 435/7.1; 536/23.1

(57) ABSTRACT

The invention relates to methods and compositions for the construction of envelope virus libraries expressing candidate proteins and the use of these libraries to identify candidate proteins and the nucleic acids encoding.

This patent application claims the benefit of the filing date of U.S. provisional application No. 60/222,697, filed Aug. 2, 2000.

### FIELD OF THE INVENTION

The invention relates to methods and compositions for the construction of envelope virus libraries expressing candidate proteins and the use of these libraries to identify candidate proteins and the nucleic acids encoding them.

### BACKGROUND OF THE INVENTION

Estimates of the total complement of human genes vary from as low as 40,000 to above 160,000. The human genome is thought to encode anywhere between 300,000 and 7.5 million protein and peptide forms (Li, M. (2000) Nature Biotechnology, 18:1251-1256). But these numbers may be significantly underestimated because even for the immunoglobulin gene—one single-gene locus containing hundreds of various V, D, and J segments—the immune system is capable of producing more than 10 million different proteins simply through gene rearrangement.

Efforts to analyze cellular protein content and function on a global scale thus require technologies that can cope with the tremendous diversity of proteins in a high-throughput format. Biomolecular display technologies, which allow the construction of a large pool of modularly coded biomolecules, their display for property selection, and rapid characterization (decoding) of their structures, are particularly useful for accessing and analyzing protein diversity on a large scale.

To date, display technologies have comprised two major groups: biological display systems that employ biological host/biological reactions, and non biological display systems that use chemical and engineering techniques. Regardless of the format, a display library consists of modularly coded molecules, each of which contains three components: displayed entities, a common linker, and the corresponding individualized codes. Over the past decade, many display formats have been developed and applied in biological and pharmaceutical research. These technologies use different types of displayed entities, linkage formats, and coding strategies.

Display libraries vary widely in size and complexity. On the basis of theoretical calculation as well as experimental results, these parameters essentially determine the probability and quality of identified biomolecules (Perelson and Oster, (1979) J. Theor. Biol., 81:645-670). Although increasing both the size and the complexity of a display library is an important objective, the fundamental aim is to optimize the assembly of building blocks that potentially lead to novel and more diverse properties.

Biological display exploits the cellular biosynthesis machinery to assemble biopolymers, the sequence of which ultimately specifies structure and distinct properties. Although nucleotide polymers, such as RNA/DNA aptamers, have yielded interesting molecules (Patel and Suir, (2000) J. Biotechnol., 74:39-60), the most commonly exploited for biological display is the nucleic acid coded synthesis of L-amino acid polymers (i.e., proteins). Most biological display systems use the 20 natural L-amino acids as building blocks and take advantage of enzymatic protein synthesis. It is now also possible to achieve template based incorporation of unnatural building blocks, such as synthetic amino acid derivatives (Cornish, V. W., et al., (1994) Proc. Natl. Acad. Sci. USA, 91:2910-2914).

One of the most important characteristics of display technologies is the ability to determine the structure of a desired compound rapidly after initial screening. Structural or sequence characterization is often accomplished by a process commonly known as coding and decoding, which can be achieved via a coupled amplification and purification process. In a biological display, chemical entities are linked to codes that have chemical and physical properties that can be readily determined, such as the sequence of nucleic acids (Brenner, S. and Lerner, R. A. (1992) Proc. Natl. Acad. Sci. USA, 89:5381-5383). A linker is used to establish the modularly coded biomolecular units, each of which possesses a unique property for either detection or deconvolution because of the attached codes. In most cases, the linkage between the displayed entity and the corresponding code is achieved by physical connections via either covalent or non covalent chemical binding.

Three types of coding formats are commonly used: peptide-on-DNA/RNA display, viral (phage) display, and cell-based display. The first of these formats uses protein-DNA/RNA complexes as its foundation. By expressing the peptide in a form that is capable of binding to its coding DNA/RNA, one can screen a large pool of complexes and identify bound peptides by the isolation and sequencing of nucleotide sequences of either DNA or RNA.

The second type of display format, viral (or phage) display, is one of the most commonly used (Smith, G. P, (1985) Science, 228:1315-1317; Dulbecco, R., U.S. Pat. No. 4,593,002; Ladner, R. C., et al., U.S. Pat. No. 5,837,500; Ladner, R. C., et al., U.S. Pat. No. 5,223,409; Dower, et al., U.S. Pat. No. 5,427,908; Russell et al., U.S. Pat. No. 5,723,287; Li U.S. Pat. No. 6,190,856). This system takes advantage of anr understanding of the coat proteins of viruses, mostly bacterial viruses (phages). For example, by cloning random oligonucleotides into the coding sequence of viral coat proteins, a library of viruses, each carrying a distinct peptide sequence as part of the coat protein, is produced. The coding information is the corresponding DNA sequence embedded in the viral genome that is packed inside the viral capsid. Several different viral systems have been used to display peptides, including lysogenic filamentous phages (Smith, G. P, (1985) Science, 228:1315-1317) and lytic lambda phage (Santini, C., et al., (1998) J. Mol. Biol., 282:125-135; Sternberg, N. and Hoess, R. H. (1995) Proc. Natl. Acad. Sci. USA, 92:1609-1613; Maruyama, I. N., et al. (1994) Proc. Natl. Acad. Sci. USA, 91:8273-8277; and Dunn, I. S., (1995) J. Mol. Biol., 248:497-506), T7 bacteriophage (Rosenberg, A., et al. (1996) Innovations 6:1-6) and T4 bacteriophage (Ren, Z. J., et al. (1996) Protein Sci., 5:1833-1843; Efimov, V. P., et al. (1995) Virus Genes 10:173-177). Lysogenic filamentous phage remains the most commonly used phage display system.

Finally, a cell-based display can be used to display large cDNA libraries in mammalian cells. This system requires efficient gene transfer, such as through the use of a viral vector. The cellular host is ( used to establish the link between the coding DNA and the displayed peptides/proteins.

Although display technology has a number of advantages, one disadvantage of display technologies that do not use membrane bound (i.e. cell display) systems, is the inability to display membrane-associated proteins in a bilayer membrane environment where they will function correctly. For example, displaying a membrane protein in a phage display system requires the construction of a fusion protein comprising a non viral membrane protein and a viral coat protein. Recruitment of the fusion protein as part of the viral coat results in the display of the protein on the surface of the viral coat. Because bacterial phages, such as the commonly used filamentous phages have a protein coat, but lack a bilayer lipid membrane, conventional phage display systems are unsatisfactory for displaying membrane proteins. Therefore, it is important to identify a display technology that will enable membrane proteins to be displayed in their native state, rather than being displayed as a fusion protein where the presence of an additional covalently attached peptide may interfere with the biological function of the displayed protein.

Accordingly, there remains a need to develop a display technology for membrane associated proteins.

It is an objective of the present invention to display membrane proteins in a modular display format on the surface of a bilayer membrane rather than as a component of a viral coat protein or viral envelope protein.

### SUMMARY OF THE INVENTION

In accordance with the objects outlined above, the present invention provides methods for generating libraries of fusion nucleic acids comprising providing a nucleic acid encoding an envelope virus genome and a nucleic acid encoding a candidate protein; the nucleic acid encoding the candidate protein is not fused to a viral glycoprotein gene.

In an additional aspect, the nucleic acid sequence encoding the candidate protein is fused, either directly or indirectly, to a nucleic acid sequence encoding a membrane anchoring sequence. The membrane anchoring sequence may be exogenous or endogenous.

In a further aspect, the invention provides for cellular and viral particle libraries comprising a nucleic acid encoding an envelope viral genome, a nucleic acid sequence encoding a candidate protein, and a nucleic acid sequence encoding a membrane anchoring sequence.

In an additional aspect, the invention provides methods of screening comprising providing a library of envelope virus particles. Each virus particle comprises a fusion nucleic acid comprising a nucleic acid sequence encoding an envelope viral genome, a nucleic acid sequence encoding a candidate protein, and a nucleic acid sequence encoding a membrane anchoring sequence. The fusion nucleic acid sequences are expressed under conditions whereby a library of viral particles are formed expressing a candidate protein on the surface of the viral envelope. At least one test molecule is added to the library of virus particles and the binding of the candidate protein is determined.

### BRIEF DESCRIPTION OF THE DRAWINGS

FIG. 1 depicts three possible configurations for expressing a candidate protein on the surface of a viral envelope: (1) it is anchored independently from viral envelope protein; (2) it is anchored via specific, non-covalent interaction with viral envelope protein, such interaction may or may not involve another protein; (3) it is anchored as one complex with viral envelope protein.

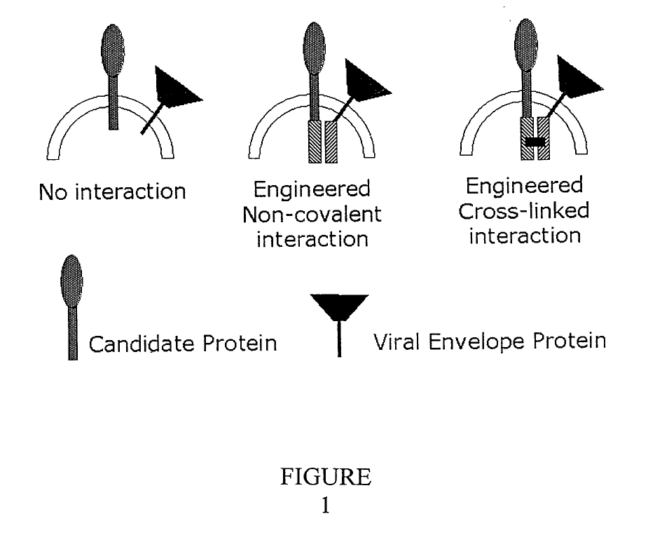

FIG. 2 depicts the recruitment of the candidate protein on the surface of the retroviral envelope during the budding process.

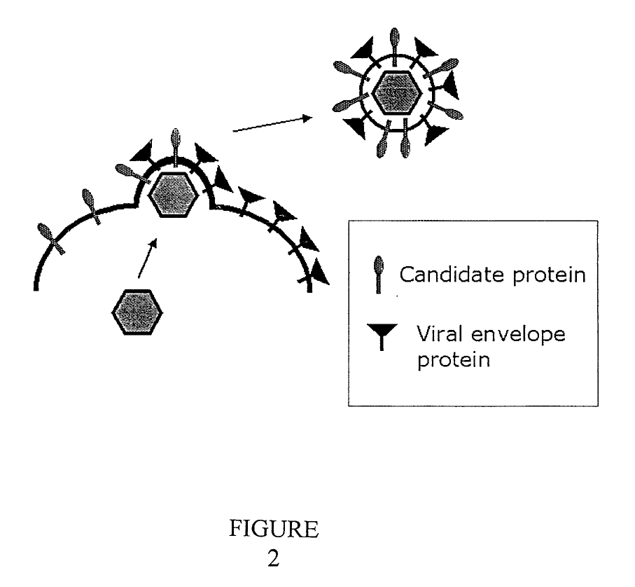

### DETAILED DESCRIPTION OF THE INVENTION

Significant effort is being channeled into display technologies that can identify proteins relevant in signaling pathways and disease states. These techniques rely on the creation of libraries comprising modular macromolecular particles that connect displayed “entities”, such as candidate proteins or peptides, with a “code”, such as a nucleic acid. The construction and screening of these modular macromolecular libraries allows for the identification of molecular entities with previously unknown functions. One of the problems facing display technologies is the difficulty of identifying membrane-associated proteins due to the lack of a bilayer membrane environment in a typical display system.

The present invention is directed to a novel method that allows for the display of a protein on the surface of bilayer membrane by inserting the nucleic acid that encodes for the protein into an envelope virus genome. The disclosed method is conceptually distinct from prior display technologies based on viral particles in that it does not require the non viral nucleic acid to be fused to a viral protein to form an open reading frame.

The system of the present invention relies on the biology of envelope viruses for the expression of nonviral proteins on the surface of virus particles. The envelope of most enveloped viruses is derived by budding from a host cell membrane and comprises preexisting lipids and other membrane components of the host cell modified by insertion of viral proteins (Coffin, J. M., (1991) In Fundamental Virology, Chapter 27, ed. by B. N. Fields, et al., Raven Press, New York, pp 645-708). In addition, spontaneous incorporation of nonviral proteins occurs (Gelderblom, et al., (1987) Z Naturforsch, 42:1328-1334; Schols, et al., (1992) Virology, 189:374-376). While the exact mechanism and preference for the expression and recruitment of non viral proteins on a viral envelope is not known, the present invention incorporates methods, including but not limited to, elevating the expression of nonviral proteins in order to favor their incorporation into the viral envelope.

Thus, nucleic acids encoding proteins of interest are linked to nucleic acid sequences encoding all of or a portion of an enveloped virus genome. Expression of the resulting “fusion nucleic acid molecule” results in the formation of an enveloped virus particle expressing the candidate protein as part of the viral envelope. The resulting library of viral particles, each displaying a distinct candidate protein, may be screened for specific binding affinity to a target molecule of interest. After screening, candidate proteins that exhibit the desired properties can be quickly pulled out using a variety of methods such as PCR amplification and the nucleic acid molecule encoding the candidate protein quickly identified.

Accordingly, the present invention provides libraries of nucleic acid molecules comprising nucleic acid sequences encoding fusion nucleic acids encoding a candidate protein and a nucleic acid that confers the ability to be packaged into an envelope virus. In addition, additional nucleic acid sequences may be included, such as nucleic acid sequences that confer the ability of the candidate peptide to be associated with the cellular membrane. For example, the candidate proteins may comprise a membrane anchoring sequence in the wild-type state, e.g. libraries of membrane proteins are used in the present invention. Alternatively, fusion partners, such as membrane anchoring sequences can be fused to the candidate proteins.

In general, as more fully outlined below, a number of different libraries can be generated using the methods of the invention. Libraries comprising all or a subset of nucleic acid sequences encoding candidate proteins can be made in a number of ways. For example, candidate proteins can be derived from cDNA libraries, genomic libraries, random libraries, constructed libraries, or generated computationally. Nucleic acid sequences encoding the candidate proteins may be linked to viral genes to form libraries of fusion nucleic acids. The fusion nucleic acid libraries can be introduced into envelope viruses to form primary and secondary libraries of virus particles expressing candidate proteins. Libraries of envelope virus particles may be introduced into host cells to generate cellular libraries containing the candidate proteins. Thus, libraries comprising fusion nucleic acids, cellular libraries and viral particle libraries may be generated using the methods of the invention.

Accordingly, the present invention provides fusion nucleic acids. By “nucleic acid” or “oligonucleotide” or grammatical equivalents herein means at least two nucleotides-covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 11 1:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); those with bicyclic structures including locked nucleic acids, Koshkin et al., J. Am. Chem. Soc. 120:13252-3 (1998); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Left. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of ETMs, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc, although generally occurring bases are preferred. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

Thus, the present invention provides libraries of nucleic acid molecules comprising nucleic acid sequences encoding fusion nucleic acids. By “fusion nucleic acid” herein is meant a plurality of nucleic acid components that are joined together, either directly or indirectly. The fusion nucleic acids may encode fusion polypeptides. By “fusion polypeptide” or “fusion peptide” or grammatical equivalents herein is meant a protein composed of a plurality of protein components, that while typically unjoined in their native state, are joined by their respective amino and carboxyl termini through a peptide linkage to form a single continuous polypeptide. Plurality in this context means at least two, and preferred embodiments generally utilize two components. It will be appreciated that the protein components can be joined directly or joined through a peptide linker/spacer as outlined below. As outlined below, additional components such as fusion partners, including targeting sequences, etc. may be included in the fusion polypeptides of the invention.

Preferably, fusion polypeptides comprising a candidate protein and a suitable fusion partner, such as a membrane anchoring sequence, are made using the methods described herein. Generally, it is not preferred to have fusion polypeptides comprising a candidate protein and a viral glycoprotein gene. As outlined herein, the fusion polypeptides do not display the candidate proteins as fusions with viral genes; rather, the candidate proteins are made and expressed in a host membrane and incorporated into the viral particle via the “budding” mechanism.

In a preferred embodiment, the fusion polypeptide comprising a candidate protein does not include a viral glycoprotein gene.

In a preferred embodiment, the fusion polypeptide comprising a candidate protein does not include a viral gene.

In a preferred embodiment, the fusion polypeptide comprising a candidate protein does not include a viral structural gene.

In a preferred embodiment, the fusion polypeptide comprising a candidate protein does not include a viral non-structural gene.

In a preferred embodiment, the fusion polypeptide comprising a candidate protein does not include a viral coat protein

The fusion nucleic acids of the invention comprise a nucleic acid encoding an envelope virus genome.

By “envelope virus genome” herein is meant the nucleic acid genome from one of several viruses characterized by the presence of an envelope. All or a portion of the genome from an envelope virus may be used in the methods of the invention. If only a portion of the genome is used, the portion used preferably contains the sequences or part of a sequence necessary to be packaged into a viral particle. Although the ability of the virus to infect a host is preferred, infectivity is not necessary for practicing the methods of the invention.

In some embodiments, the viral genome, by itself is not sufficient for packaging. For example, retroviral particles can be generated in cell lines containing packaging functions, such as cell lines expressing one or more of the gag, pol or env genes. In this embodiment, what is important is that the candidate protein is on the surface of the viral particle and is encoded in a fusion nucleic acid with the remaining viral genes.

By “envelope” or “membrane” herein is meant the lipid bilayer surrounding the “nucleocapsid” or “core”. Most enveloped viruses acquire their membrane or envelope by budding through an appropriate cellular membrane—the plasma membrane in many cases, the endoplasmic reticulum (ER), Golgi or nuclear membrane in other cases. Enveloped viruses use the cell's compartmentalization mechanisms to direct the insertion of their surface glycoproteins into the cell membrane. Although all of the events leading to the formation of a mature virus particle are not completely understood, in some enveloped viruses there appears to be a transmembrane interaction of the membrane glycoproteins and the components of the virus in the cytoplasm, followed by a pinching off from the cell surface or into the lumen of the ER or Golgi. The lipids in the resulting bilayer derive from the cell, whereas the majority of the proteins are virally encoded (Harrison, S. C. (1996) Chapter 3, “Virus Structure”, In Fields Virology, 3rd ed., Vol 1, Lippincott-Raven, Philadelphia, pp. 59-99).

The source of the viral genome may be from any family of enveloped viruses, including RNA and DNA viruses. Single stranded RNA enveloped virus families include: Togaviridae; Flaviviridae; Coronaviridae (including the floating genus Arterivirus); virus families belonging to the order Mononegavirales (i.e., Paramyxoviridae, Rhabdoviridae, Filoviridae); Orthomyxoviridae; Bunyaviridae; Arenaviridae; and, Retroviridae. Double stranded and double stranded/single stranded DNA enveloped virus families include: Hepadnaviridae; Herpesviridae; Poxviridae; Iridoviridae; and the family of unnamed viruses including, but not limited to African swine fever virus (Murphy, F. A. chapter 2, “Virus Taxonomy”, In Fields Virology vol 1, 3rd edition, Lippincott-Raven, Philadelphia, pp. 15-57).

In a preferred embodiment, envelope viruses belonging to the family Retroviridae are used in the methods of the invention. Included within the family Retroviridae are: the Avain Leukosis-Sarcina Virus (ALSV) group; the mammalian C-type virus group; the B-type virus group; the D-type virus group; the HTLV-BLV group, the Lentivitinae (which includes human immunodeficiency virus (HIV)) and the Spumavirinae.

In a preferred embodiment, vector constructs based on retroviral genomes are used. See for example, Russell et al., U.S. Pat. No. 5,723,287; Strehlow, D., et al (2000) Proc. Natl. Acad. Sci. USA, 97:42094214Zerangue, N., et al., (2001) Proc. Natl. Acad. Sci. USA, 98:2431-2436; Retroviral Expression Systems, Clontech; all of which are expressly incorporated herein by reference. Preferably, such a construct would include the following components: an expression cassette for the insertion of nucleic acid sequences encoding candidate proteins; unique, non- complementary restriction sites flanking the sequences which encode the candidate protein; a bacterial plasmid origin of replication and antibiotic marker; long terminal repeat sequences; a tRNA primer binding site; and, a polypurine tract. Additional components included fusion partners, such as dimerization sequences that facilitate association of the candidate protein with a viral protein, GPI anchor signal sequences, labels; IRES, etc., are described below.

The fusion nucleic acids further comprise nucleic acid encoding a candidate protein. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acd. Sci. U.S.A. 89(20:9367-71 (1992)], generally depending on the method of synthesis. Thus “amino acid”, or “peptide residue”, as used herein means both naturally occurring and synthetic amino acids. For example, homo-phenylalanine, citrulline, and noreleucine are considered amino acids for the purposes of the invention. “Amino acid” also includes imino acid residues such as proline and hydroxyproline. In addition, any amino acid representing a component of the variant proteins of the present invention can be replaced by the same amino acid but of the opposite chirality. Thus, any amino acid naturally occurring in the L-configuration (which may also be referred to as the R or S, depending upon the structure of the chemical entity) may be replaced with an amino acid of the same chemical structural type, but of the opposite chirality, generally referred to as the D-amino acid but which can additionally be referred to as the R- or the S-, depending upon its composition and chemical configuration. Such derivatives have the property of greatly increased stability, and therefore are advantageous in the formulation of compounds which may have longer in vivo half lives, when administered by oral, intravenous, intramuscular, intraperitoneal, topical, rectal, intraocular, or other routes.

In the preferred embodiment, the amino acids are in the (S) or L-configuration. If non-naturally occurring side chains are used, non-amino acid substituents may be used, for example to prevent or retard in vivo degradations. Proteins including non-naturally occurring amino acids may be synthesized or in some cases, made recombinantly; see van Hest et al., FEBS Lett 428:(1-2) 68-70 May 22 1998 and Tang et al., Abstr. Pap Am. Chem. S218:U138-U138 Part 2 Aug. 22, 1999, both of which are expressly incorporated by reference herein.

The candidate proteins of the present invention may be from prokaryotes and eukaryotes, such as bacteria (including extremeophiles such as the archebacteria), fungi, insects, fish, and mammals Suitable mammals include, but are not limited to, rodents (rats, mice, hamsters, guinea pigs, etc.), primates, farm animals (including sheep, goats, pigs, cows, horses, etc) and in the most preferred embodiment, from humans.

The nucleic acid sequences may be naturally occurring nucleic acids, such as cDNAs or genomic DNAs, random nucleic acids, or “biased” random nucleic acids.

Suitable candidate proteins include, but are not limited to, industrial, pharmaceutical, and agricultural proteins, including ligands, cell surface receptors, antigens, antibodies, cytokines, hormones, transcription factors, signaling modules, cytoskeletal proteins and enzymes. Suitable classes of enzymes include, but are not limited to, hydrolases such as proteases, carbohydrases, lipases; isomerases such as racemases, epimerases, tautomerases, or mutases; transferases, kinases, oxidoreductases, and phophatases. Suitable enzymes are listed in the Swiss-Prot enzyme database. Suitable protein backbones include, but are not limited to, all of those found in the protein data base compiled and serviced by the Research Collaboratory for Structural Bioinformatics (RCSB, formerly the Brookhaven National Lab).

Specifically, preferred pharmaceutical candidate proteins include, but are not limited to, those with known structures (including variants) including cytokines (IL-1 ra (+receptor complex), IL-1(receptor alone), IL-1a, IL-1b (including variants and or receptor complex), IL-2, IL-3, IL4, IL-5, IL-6, IL-8, IL-10, IFN-β, INF-γ, IFN-α-2a; IFN-α-2B, TNF-α; CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration Inhibitory Factor, Human Glycosylation-Inhibiting Factor, Human Rantes, Human Macrophage Inflammatory Protein 1 Beta, human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor A, Vascular Endothelial growth factor (VEGF), acidic Fibroblast growth factor, basic Fibroblast growth factor, Endothelial growth factor, Nerve growth factor, Brain Derived Neurotrophic Factor, Ciliary Neurotrophic Factor, Platelet Derived Growth Factor, Human Hepatocyte Growth Factor, Glial Cell-Derived Neurotrophic Factor, (as well as the 55 cytokines in PDB Jan. 12, 1999)); urokinase; Erythropoietin; other extracellular signalling moeities, including, but not limited to, hedgehog Sonic, hedgehog Desert, hedgehog Indian, hCG; coaguation factors including, but not limited to, TPA and Factor VIIa; transcription factors, including but not limited to, p53, p53 tetramerization domain, Zn fingers (of which more than 12 have structures), homeodomains (of which 8 have structures), leucine zippers (of which 4 have structures); antibodies, including, but not limited to, cFv; viral proteins, including, but not limited to, hemagglutinin trimerization domain and hiv Gp41 ectodomain (fusion domain); intracellular signalling modules, including, but not limited to, SH2 domains (of which 8 structures are known), SH3 domains (of which 11 have structures), and Pleckstin Homology Domains; receptors, including, but not limited to, the extracellular Region Of Human Tissue Factor Cytokine-Binding Region Of Gp130, G-CSF receptor, erythropoietin receptor, Fibroblast Growth Factor receptor, TNF receptor, IL-1 receptor, IL-1 receptor/IL1ra complex, IL4 receptor, INF-γ receptor alpha chain, MHC Class I, MHC Class II, T Cell Receptor, Insulin receptor, insulin receptor tyrosine kinase and human growth hormone receptor.

Specifically, preferred industrial candidate proteins include, but are not limited to, those with known structures (including variants) including proteases, (including, but not limited to papains, subtilisins), cellulases (including , but not limited to, endoglucanases I, II, and IlI, exoglucanases, xylanases, ligninases, cellobiohydrolases I, II, and III, carbohydrases (including, but not limited to glucoamylases, α-amylases, glucose isomerases) and lipases.

Specifically, preferred agricultural candidate proteins include, but are not limited to, those with known structures (including variants) including xylose isomerase, pectinases, cellulases, peroxidases, rubisco, ADP glucose phrophosphorlyase, as well as enzymes involved in oil biosynthesis, sterol biosynthesis, carbohydrate biosynthesis, and the synthesis of secondary metabolites.

By “candidate protein” herein is meant a protein to be tested for binding, association or effect in an assay of the invention, including both in vitro (e.g. cell free systems) or ex vivo (within cells). Generally, as outline below, the candidate protein contains a transmembrane domain that is either endogenous or added as a fusion partner. As will be appreciated by those in the art, the source of the candidate protein libraries can vary, particularly depending on the end use of the system.

In a preferred embodiment, libraries of candidate proteins are used. Libraries comprising all or a subset of nucleic acid sequences encoding candidate proteins can be used in the methods of the invention. For example, candidate proteins can be derived from cDNA libraries, genomic libraries, random libraries, constructed libraries, or generated computationally. The library should provide a sufficiently structurally diverse population of randomized expression products to effect a probabilistically sufficient range of cellular responses to provide one or more cells exhibiting a desired response. Accordingly, an interaction library must be large enough so that at least one of its members will have a structure that gives it affinity for some molecule, protein, or other factor whose activity is necessary for completion of the signaling pathway. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length, as proposed here for expression in enveloped viruses such as retroviruses, has the potential to code for 20⁷ (10⁹) to 20²⁰. Thus, with libraries of 10⁷ to 10⁸ the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁸ and most preferably at least 10⁹ different expression products are simultaneously analyzed in the subject methods, although libraries of less complexity (e.g., 10², 10³, 10⁴, or 10⁵ different expression products) or greater complexity (e.g., 10¹⁰, 10¹¹, or 10¹² different expression products) are appropriate for use in the present invention. Preferred methods maximize library size and diversity.

In a preferred embodiment, the candidate proteins are derived from cDNA libraries. The cDNA libraries can be derived from any number of different cells, particularly those outlined for host cells herein, and include cDNA libraries generated from eucaryotic and procaryotic cells, viruses, cells infected with viruses or other pathogens, genetically altered cells, etc. Preferred embodiments, as outlined below, include cDNA libraries made from different individuals, such as different patients, particularly human patients. The cDNA libraries may be complete libraries or partial libraries. Furthermore, the library of candidate proteins can be derived from a single cDNA source or multiple sources; that is, cDNA from multiple cell types or multiple individuals or multiple pathogens can be combined in a screen. The cDNA library may utilize entire cDNA constructs or fractionated constructs, including random or targeted fractionation. Suitable fractionation techniques include enzymatic, chemical or mechanical fractionation. cDNA libraries enriched for a specific class of proteins, such as type I membrane proteins, also can be made (Tashiro, K., et al., (1993) Science, 261:600-603).

In a preferred embodiment, the candidate proteins are derived from genomic libraries. As above, the genomic libraries can be derived from any number of different cells, particularly those outlined for host cells herein, and include genomic libraries generated from eucaryotic and procaryotic cells, viruses, cells infected with viruses or other pathogens, genetically altered cells, etc. Preferred embodiments, as outlined below, include genomic libraries made from different individuals, such as different patients, particularly human patients. The genomic libraries may be complete libraries or partial libraries. Furthermore, the library of candidate proteins can be derived from a single genomic source or multiple sources; that is, genomic DNA from multiple cell types or multiple individuals or multiple pathogens can be combined in a screen. The genomic library may utilize entire genomic constructs or fractionated constructs, including random or targeted fractionation. Suitable fractionation techniques include enzymatic, chemical or mechanical fractionation.

In a preferred embodiment, the candidate protein library is a random or biased random peptide library. Generally, peptides ranging from about 4 amino acids in length to about 100 amino acids may be used, with peptides ranging from about 5 to about 50 being preferred, with from about 8 to about 30 being particularly preferred and from about 10 to about 25 being especially preferred.

In any library system encoded by oligonucleotide synthesis, complete control over the codons that will eventually be incorporated into the peptide structure is difficult. This is especially true in the case of codons encoding stop signals (TAA, TGA, TAG). In a synthesis with NNN as the random region, there is a 3/64, or 4.69%, chance that the codon will be a stop codon. Thus, in a peptide of 10 residues, there is a high likelihood that 46.7% of the peptides will prematurely terminate. One way to alleviate this is to have random residues encoded as NNK, where K=T or G. This allows for encoding of all potential amino acids (changing their relative representation slightly), but importantly preventing the encoding of two stop residues TAA and TGA. Thus, libraries encoding a 10 amino acid peptide will have a 15.6% chance to terminate prematurely.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, PDZ domains, serines, threonines, tyrosines or histidines for phosphorylation sites, etc., or to purines, etc.

In a preferred embodiment, the bias is towards peptides or nucleic acids that interact with known classes of molecules. For example, when the candidate protein is a peptide, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from Wasps, can be limited to a short peptide region with death-inducing apoptotic or G protein inducing functions. Magainin, a natural peptide derived from Xenopus, can have potent anti-tumour and anti-microbial activity. Short peptide fragments of a protein kinase C isozyme (βPKC), have been shown to block nuclear translocation of βPKC in Xenopus oocytes following stimulation. And, short SH-3 target peptides have been used as pseudosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate proteins as well.

Thus, a number of molecules or protein domains are suitable as starting points for the generation of biased randomized candidate proteins. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, Traf, etc. Similarly, there are a number of known nucleic acid binding proteins containing domains suitable for use in the invention. For example, leucine zipper consensus sequences are known.

In a preferred embodiment, biased SH-3 domain-binding oligonucleotides/peptides are made. SH-3 domains have been shown to recognize short target motifs (SH-3 domain-binding peptides), about ten to twelve residues in a linear sequence, that can be encoded as short peptides with high affinity for the target SH-3 domain. Consensus sequences for SH-3 domain binding proteins have been proposed. Thus, in a preferred embodiment, oligos/peptides are made with the following biases

1. XXXPPXPXX, wherein X is a randomized residue.

2. (within the positions of residue positions 11 to -2):

```
            11 10 9 8 7 6 5 4 3 2 1
Met Glyaa11aa10 aa9 aa8 aa7 Arg Pro Leu Pro Pro hyd
  0 −1 −2
Pro hyd hyd Gly Gly Pro Pro STOP
atg ggc nnk nnk nnk nnk nnk aga cct ctg cct cca sbk ggg sbk sbk gga ggc cca cct TAA1.
```

In this embodiment, the N-terminus flanking region is suggested to have the greatest effects on binding affinity and is therefore entirely randomized. “Hyd” indicates a bias toward a hydrophobic residue, i.e.- Val, Ala, Gly, Leu, Pro, Arg. To encode a hydrophobically biased residue, “sbk” codon biased structure is used. Examination of the codons within the genetic code will ensure this encodes generally hydrophobic residues. s=g,c; b=t, g, c; v=a, g, c; m=a, c; k=t, g; n=a, t, g, c.

In addition, the candidate protein library may be a constructed library; that is, it may be built to contain only members of a defined class, or combinations of classes. For example, libraries of soluble proteins or membrane proteins may be built; libraries of membrane anchoring sequences may be built, libraries of immunoglobulins may be built, or libraries of G-protein coupled receptors, tumor suppressor genes, proteases, transcription factors, phosphotases, kinases, etc.

In a preferred embodiment the candidate protein library is a constructed library containing membrane proteins. By “membrane protein” herein is meant any protein that is membrane-bound. Included within this definition of membrane protein are integral membrane proteins; peripheral membrane proteins; and membrane anchoring proteins, such as hydrophic peptide mediated anchoring proteins or lipid mediated anchoring proteins (i.e. GPI). The membrane proteins may have a single transmembrane domain or multiple transmembrane domains (i.e. such as the commonly used type of structure seen in many serpentine transmembrane proteins that involves 7 hydrophobic domains inserted into the plasma membrane separated by hydrophilic regions that are looped out alternatively into either the cytoplasm or the extracellular space). In addition computer programs may be used to identify transmembrane domains. Also included within the definition of membrane proteins are membrane proteins that exist as oligomers (either homo-oligomers or hetero-oligomers) even though only some of the proteins comprising the oligomeric complex are not membrane bound. An example of an oligomeric complex in which only some of the proteins are membrane bound are the G-protein coupled 7-transmembrane receptors. Sources of membrane proteins include, but are not limited to, the plasma membrane, organelle membranes such as the ER, Golgi, and the nuclear membrane. Membrane proteins may also include extracellular domains that are retained on the surface of the membrane via a number of mechanisms, such as through interactions with other proteins or protein factors or due to the native composition of the extracellular domain itself, or, as outlined herein, by fusion with membrane anchoring sequences.

Alternatively, the candidate protein library is a constructed library containing soluble proteins. By “soluble protein” herein is meant any protein that is not membrane bound, including intracellular, nuclear and secreted proteins. Suitable soluble proteins include pharmaceutical proteins, industrial proteins and agricultural proteins as defined above. In some embodiments, the soluble protein may be anchored to the envelope viral surface using the methods described herein such as fusion with a membrane anchoring sequence or using methods known by those of skill in the art.

In other embodiments the candidate protein library is a constructed library containing membrane anchoring sequences as defined herein. In addition, computational methods as described herein may be used to generate variant membrane anchoring sequences.

In alternative embodiments, the candidate proteins may be oriented in such a manner that the protein is not exposed on the surface of the viral envelope. For example, a membrane anchoring sequence may be used to attach the candidate protein to the inner side of the viral envelope, such that the protein is oriented toward the interior, i.e. “cytoplasmic interior”, of the viral particle.

In a preferred embodiment, a computational method is used to generate the candidate protein library. Preferably the method is Protein Design Automation (PDA), as is described in U.S. Ser. Nos. 60/061,097, 60/043,464, 60/054,678, 09/127,926 and PCT US98/07254, all of which are expressly incorporated herein by reference. Briefly, PDA can be described as follows. A known protein structure is used as the starting point. The residues to be optimized are then identified, which may be the entire sequence or subset(s) thereof. The side chains of any positions to be varied are then removed. The resulting structure consisting of the protein backbone and the remaining sidechains is called the template.

Each variable residue position is then preferably classified as a core residue, a surface residue, or a boundary residue; each classification defines a subset of possible amino acid residues for the position (for example, core residues generally will be selected from the set of hydrophobic residues, surface residues generally will be selected from the hydrophilic residues, and boundary residues may be either). Each amino acid can be represented by a discrete set of all allowed conformers of each side chain, called rotamers. Thus, to arrive at an optimal sequence for a backbone, all possible sequences of rotamers must be screened, where each backbone position can be occupied either by each amino acid in all its possible rotameric states, or a subset of amino acids, and thus a subset of rotamers.

Two sets of interactions are then calculated for each rotamer at every position: the interaction of the rotamer side chain with all or part of the backbone (the “singles” energy, also called the rotameritemplate or rotamer/backbone energy), and the interaction of the rotamer side chain with all other possible rotamers at every other position or a subset of the other positions (the “doubles” energy, also called the rotamer/rotamer energy). The energy of each of these interactions is calculated through the use of a variety of scoring functions, which include the energy of van der Waal's forces, the energy of hydrogen bonding, the energy of secondary structure propensity, the energy of surface area solvation and the electrostatics. Thus, the total energy of each rotamer interaction, both with the backbone and other rotamers, is calculated, and stored in a matrix form.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, a “Dead End Elimination” (DEE) calculation is performed. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably. DEE can be rerun comparing pairs of rotamers, or combinations of rotamers, which will eventually result in the determination of a single sequence which represents the global optimum energy.

Once the global solution has been found, a Monte Carlo search may be done to generate a rank-ordered list of sequences in the neighborhood of the DEE solution. Starting at the DEE solution, random positions are changed to other rotamers, and the new sequence energy is calculated. If the new sequence meets the criteria for acceptance, it is used as a starting point for another jump. After a predetermined number of jumps, a rank-ordered list of sequences is generated. Monte Carlo searching is a sampling technique to explore sequence space around the global minimum or to find new local minima distant in sequence space. As is more additionally outlined below, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Similarly, for all the sampling techniques, the acceptance criteria of whether a sampling jump is accepted can be altered.

As outlined in U.S. Ser. No. 09/127,926, the protein backbone (comprising (for a naturally occurring protein) the nitrogen, the carbonyl carbon, the α-carbon, and the carbonyl oxygen, along with the direction of the vector from the α-carbon to the β-carbon) may be altered prior to the computational analysis, by varying a set of parameters called supersecondary structure parameters.

Once a protein structure backbone is generated (with alterations, as outlined above) and input into the computer, explicit hydrogens are added if not included within the structure (for example, if the structure was generated by X-ray crystallography, hydrogens must be added). After hydrogen addition, energy minimization of the structure is run, to relax the hydrogens as well as the other atoms, bond angles and bond lengths. In a preferred embodiment, this is done by doing a number of steps of conjugate gradient minimization (Mayo et a., J. Phys. Chem. 94:8897 (1990)) of atomic coordinate positions to minimize the Dreiding force field with no electrostatics. Generally from about 10 to about 250 steps is preferred, with about 50 being most preferred.

The protein backbone structure contains at least one variable residue position. As is known in the art, the residues, or amino acids, of proteins are generally sequentially numbered starting with the N-terminus of the protein. Thus a protein having a methionine at it's N-terminus is said to have a methionine at residue or amino acid position 1, with the next residues as 2, 3, 4, etc. At each position, the wild type (i.e. naturally occurring) protein may have one of at least 20 amino acids, in any number of rotamers. By “variable residue position” herein is meant an amino acid position of the protein to be designed that is not fixed in the design method as a specific residue or rotamer, generally the wild-type residue or rotamer.

In a preferred embodiment, all of the residue positions of the protein are variable. That is, every amino acid side chain may be altered in the methods of the present invention. This is particularly desirable for smaller proteins, although the present methods allow the design of larger proteins as well. While there is no theoretical limit to the length of the protein which may be designed this way, there is a practical computational limit.

In an alternate preferred embodiment, only some of the residue positions of the protein are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable (for example, to eliminate a proteolytic site or alter the substrate specificity of an enzyme), the residue may be fixed as a particular amino acid. Alternatively, the methods of the present invention may be used to evaluate mutations de novo, as is discussed below. In an alternate preferred embodiment, a fixed position may be “floated”; the amino acid at that position is fixed, but different rotamers of that amino acid are tested. In this embodiment, the variable residues may be at least one, or anywhere from 0.1% to 99.9% of the total number of residues- Thus, for example, it may be possible to change only a few (or one) residues, or most of the residues, with all possibilities in between.

In a preferred embodiment, residues which can be fixed include, but are not limited to, structurally or biologically functional residues; alternatively, biologically functional residues may specifically not be fixed. For example, residues which are known to be important for biological activity, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, metal binding sites, critical hydrogen bonding residues, residues critical for backbone conformation such as proline or glycine, residues critical for packing interactions, etc. may all be fixed in a conformation or as a single rotamer, or “floated”.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

In a preferred embodiment, each variable position is classified as either a core, surface or boundary residue position, although in some cases, as explained below, the variable position may be set to glycine to minimize backbone strain. In addition, as outlined herein, residues need not be classified, they can be chosen as variable and any set of amino acids may be used. Any combination of core, surface and boundary positions can be utilized: core, surface and boundary residues; core and surface residues; core and boundary residues, and surface and boundary residues, as well as core residues alone, surface residues alone, or boundary residues alone.

The classification of residue positions as core, surface or boundary may be done in several ways, as will be appreciated by those in the art. In a preferred embodiment, the classification is done via a visual scan of the original protein backbone structure, including the side chains, and assigning a classification based on a subjective evaluation of one skilled in the art of protein modeling. Alternatively, a preferred embodiment utilizes an assessment of the orientation of the Cα-Cβ vectors relative to a solvent accessible surface computed using only the template Cα atoms, as outlined in U.S. Ser. Nos. 601061,097, 60/043,464, 60/054,678, 09/127,926 and PCT US98/07254. Alternatively, a surface area calculation can be done.

Once each variable position is classified as either core, surface or boundary, a set of amino acid side chains, and thus a set of rotamers, is assigned to each position. That is, the set of possible amino acid side chains that the program will allow to be considered at any particular position is chosen. Subsequently, once the possible amino acid side chains are chosen, the set of rotamers that will be evaluated at a particular position can be determined. Thus, a core residue will generally be selected from the group of hydrophobic residues consisting of alanine, valine, isoleucine, leucine, phenylalanine, tyrosine, tryptophan, and methionine (in some embodiments, when the a scaling factor of the van der Waals scoring function, described below, is low, methionine is removed from the set), and the rotamer set for each core position potentially includes rotamers for these eight amino acid side chains (all the rotamers if a backbone independent library is used, and subsets if a rotamer dependent backbone is used). Similarly, surface positions are generally selected from the group of hydrophilic residues consisting of alanine, serine, threonine, aspartic acid, asparagine, glutamine, glutamic acid, arginine, lysine and histidine. The rotamer set for each surface position thus includes rotamers for these ten residues. Finally, boundary positions are generally chosen from alanine, serine, threonine, aspartic acid, asparagine, glutamine, glutamic acid, arginine, lysine histidine, valine, isoleucine, leucine, phenylalanine, tyrosine, tryptophan, and methionine. The rotamer set for each boundary position thus potentially includes every rotamer for these seventeen residues (assuming cysteine, glycine and proline are not used, although they can be). Additionally, in some preferred embodiments, a set of 18 naturally occurring amino acids (all except cysteine and proline, which are known to be particularly disruptive) are used.

Thus, as will be appreciated by those in the art, there is a computational benefit to classifying the residue positions, as it decreases the number of calculations. It should also be noted that there may be situations where the sets of core, boundary and surface residues are altered from those described above; for example, under some circumstances, one or more amino acids is either added or subtracted from the set of allowed amino acids. For example, some proteins which dimerize or multimerize, or have ligand binding sites, may contain hydrophobic surface residues, etc. In addition, residues that do not allow helix “capping” or the favorable interaction with an a-helix dipole may be subtracted from a set of allowed residues. This modification of amino acid groups is done on a residue by residue basis.

In a preferred embodiment, proline, cysteine and glycine are not included in the list of possible amino acid side chains, and thus the rotamers for these side chains are not used. However, in a preferred embodiment, when the variable residue position has a 4) angle (that is, the dihedral angle defined by 1) the carbonyl carbon of the preceding amino acid; 2) the nitrogen atom of the current residue; 3) the α-carbon of the current residue; and 4) the carbonyl carbon of the current residue) greater than 0°, the position is set to glycine to minimize backbone strain.

Once the group of potential rotamers is assigned for each variable residue position, processing proceeds as outlined in U.S. Ser. No. 09/127,926 and PCT US98/07254. This processing step entails analyzing interactions of the rotamers with each other and with the protein backbone to generate optimized protein sequences. Simplistically, the processing initially comprises the use of a number of scoring functions to calculate energies of interactions of the rotamers, either to the backbone itself or other rotamers. Preferred PDA scoring functions include, but are not limited to, a Van der Waals potential scoring function, a hydrogen bond potential scoring function, an atomic solvation scoring function, a secondary structure propensity scoring function and an electrostatic scoring function. As is further described below, at least one scoring function is used to score each position, although the scoring functions may differ depending on the position classification or other considerations, like favorable interaction with an α-helix dipole. As outlined below, the total energy which is used in the calculations is the sum of the energy of each scoring function used at a particular position, as is generally shown in Equation 1:

Eₜₒₜₐₗ=nE_{vdw}+nEₐₛ+nE_{h-bonding}+nEₛₛ+nE_{elec} Equation 1

In Equation 1, the total energy is the sum of the energy of the van der Waals potential (E_{vdw}), the energy of atomic solvation (Eₐₛ), the energy of hydrogen bonding (E_{h-bonding}), the energy of secondary structure (Eₛₛ) and the energy of electrostatic interaction (E_{elec}). The term n is either 0 or 1, depending on whether the term is to be considered for the particular residue position.

As outlined in U.S. Ser. Nos. 60/061,097, 60/043,464, 60/054,678, 09/127,926 and PCT US98/07254, any combination of these scoring functions, either alone or in combination, may be used. Once the scoring functions to be used are identified for each variable position, the preferred first step in the computational analysis comprises the determination of the interaction of each possible rotamer with all or part of the remainder of the protein. That is, the energy of interaction, as measured by one or more of the scoring functions, of each possible rotamer at each variable residue position with either the backbone or other rotamers, is calculated. In a preferred embodiment, the interaction of each rotamer with the entire remainder of the protein, i.e. both the entire template and all other rotamers, is done. However, as outlined above, it is possible to only model a portion of a protein, for example a domain of a larger protein, and thus in some cases, not all of the protein need be considered. The term “portion”, as used herein, with regard to a protein refers to a fragment of that protein. This fragment may range in size from 10 amino acid residues to the entire amino acid sequence minus one amino acid. Accordingly, the term “portion”, as used herein, with regard to a nucleic refers to a fragment of that nucleic acid. This fragment may range in size from 10 nucleotides to the entire nucleic acid sequence minus one nucleotide.

In a preferred embodiment, the first step of the computational processing is done by calculating two sets of interactions for each rotamer at every position: the interaction of the rotamer side chain with the template or backbone (the “singles” energy), and the interaction of the rotamer side chain with all other possible rotamers at every other position (the “doubles” energy), whether that position is varied or floated. It should be understood that the backbone in this case includes both the atoms of the protein structure backbone, as well as the atoms of any fixed residues, wherein the fixed residues are defined as a particular conformation of an amino acid.

Thus, “singles” (rotamer/template) energies are calculated for the interaction of every possible rotamer at every variable residue position with the backbone, using some or all of the scoring functions. Thus, for the hydrogen bonding scoring function, every hydrogen bonding atom of the rotamer and every hydrogen bonding atom of the backbone is evaluated, and the E_{HB} is calculated for each possible rotamer at every variable position. Similarly, for the van der Waals scoring function, every atom of the rotamer is compared to every atom of the template (generally excluding the backbone atoms of its own residue), and the E_{vdW} is calculated for each possible rotamer at every variable residue position. In addition, generally no van der Waals energy is calculated if the atoms are connected by three bonds or less. For the atomic solvation scoring function, the surface of the rotamer is measured against the surface of the template, and the Eₐₛ for each possible rotamer at every variable residue position is calculated. The secondary structure propensity scoring function is also considered as a singles energy, and thus the total singles energy may contain an Eₛₛ term. As will be appreciated by those in the art, many of these energy terms will be close to zero, depending on the physical distance between the rotamer and the template position; that is, the farther apart the two moieties, the lower the energy.

For the calculation of “doubles” energy (rotamer/rotamer), the interaction energy of each possible rotamer is compared with every possible rotamer at all other variable residue positions. Thus, “doubles” energies are calculated for the interaction of every possible rotamer at every variable residue position with every possible rotamer at every other variable residue position, using some or all of the scoring functions. Thus, for the hydrogen bonding scoring function, every hydrogen bonding atom of the first rotamer and every hydrogen bonding atom of every possible second rotamer is evaluated, and the E_{HB} is calculated for each possible rotamer pair for any two variable positions. Similarly, for the van der Waals scoring function, every atom of the first rotamer is compared to every atom of every possible second rotamer, and the E_{vdW} is calculated for each possible rotamer pair at every two variable residue positions. For the atomic solvation scoring function, the surface of the first rotamer is measured against the surface of every possible second rotamer, and the Eₐₛ for each possible rotamer pair at every two variable residue positions is calculated. The secondary structure propensity scoring function need not be run as a “doubles” energy, as it is considered as a component of the “singles” energy. As will be appreciated by those in the art, many of these double energy terms will be close to zero, depending on the physical distance between the first rotamer and the second rotamer; that is, the farther apart the two moieties, the lower the energy.

In addition, as will be appreciated by those in the art, a variety of force fields that can be used in the PDA calculations can be used, including, but not limited to, Dreiding I and Dreiding II (Mayo et al, J. Phys. Chem. 948897 (1990)), AMBER (Weiner et al., J. Amer. Chem. Soc. 106:765 (1984) and Weineretal., J. Comp. Chem. 106:230 (1986)), MM2 (Allinger J. Chem. Soc. 99:8127 (1977), Liljefors et al., J. Com. Chem. 8:1051 (1987)); MMP2 (Sprague et al., J. Comp. Chem. 8:581 (1987)); CHARMM (Brooks et al., J. Comp. Chem. 106:187 (1983)); GROMOS; and MM3 (Allinger et al., J. Amer. Chem. Soc. 111:8551 (1989)), OPLS-AA (Jorgensen, et al., J. Am. Chem. Soc. (1996), v 118, pp 11225-11236; Jorgensen, W. L.; BOSS, Version 4.1; Yale University: New Haven, CT (1999)); OPLS (Jorgensen, et al., J. Am. Chem. Soc. (1988), v 110, pp 1657ff; Jorgensen, et al., J Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; al., J. Comp. Chem. (1998), v 19, pp259-276; Forcefield for Protein Structure Prediction (Liwo, et al., Proc. Natl. Acad. Sci. USA (1999), v 96, pp5482-5485); ECEPP/3 (Liwo et al., J Protein Chem 1994 May;13(4):375-80); AMBER 1.1 force field (Weiner, et al., J. Am. Chem. Soc. v 106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff3.0 (Dauber-Osguthorpe, et al.,(1988) Proteins: Structure, Function and Genetics, v4,pp31-47); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference.

Once the singles and doubles energies are calculated and stored, the next step of the computational processing may occur. As outlined in U.S. Ser. No. 09/127,926 and PCT US98/07254, preferred embodiments utilize a Dead End Elimination (DEE) step, and preferably a Monte Carlo step.

PDA, viewed broadly, has three components that may be varied to alter the output (e.g. the primary library): the scoring functions used in the process; the filtering technique, and the sampling technique.

In a preferred embodiment, the scoring functions may be altered. In a preferred embodiment, the scoring functions outlined above may be biased or weighted in a variety of ways. For-example, a bias towards or away from a reference sequence or family of sequences can be done; for example, a bias towards wild-type or homolog residues may be used. Similarly, the entire protein or a fragment of it may be biased; for example, the active site may be biased towards wild-type residues, or domain residues towards a particular desired physical property can be done. Furthermore, a bias towards or against increased energy can be generated. Additional scoring function biases include, but are not limited to applying electrostatic potential gradients or hydrophobicity gradients, adding a substrate or binding partner to the calculation, or biasing towards a desired charge or hydrophobicity.

In addition, in an alternative embodiment, there are a variety of additional scoring functions that may be used. Additional scoring functions include, but are not limited to torsional potentials, or residue pair potentials, or residue entropy potentials. Such additional scoring functions can be used alone, or as functions for processing the library after it is scored initially. For example, a variety of functions derived from data on binding of peptides to MHC (Major Histocompatibility Complex) can be used to rescore a library in order to eliminate proteins containing sequences which can potentially bind to MHC, i.e. potentially immunogenic sequences.

In a preferred embodiment, a variety of filtering techniques can be done, including, but not limited to, DEE and its related counterparts. Additional filtering techniques include, but are not limited to branch-and-bound techniques for finding optimal sequences (Gordon and Majo, Structure Fold. Des. 7:1089-98, 1999), and exhaustive enumeration of sequences. It should be noted however, that some techniques may also be done without any filtering techniques; for example, sampling techniques can be used to find good sequences, in the absence of filtering.

As will be appreciated by those in the art, once an optimized sequence or set of sequences is generated, (or again, these need not be optimized or ordered) a variety of sequence space sampling methods can be done, either in addition to the preferred Monte Carlo methods, or instead of a Monte Carlo search. That is, once a sequence or set of sequences is generated, preferred methods utilize sampling techniques to allow the generation of additional, related sequences for testing.

These sampling methods can include the use of amino acid substitutions, insertions or deletions, or recombinations of one or more sequences. As outlined herein, a preferred embodiment utilizes a Monte Carlo search, which is a series of biased, systematic, or random jumps- However, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Jumps where multiple residue positions are coupled (two residues always change together, or never change together), jumps where whole sets of residues change to other sequences (e.g., recombination). Similarly, for all the sampling techniques, the acceptance criteria of whether a sampling jump is accepted can be altered, to allow broad searches at high temperature and narrow searches close to local optima at low temperatures. See Metropolis et al., J. Chem Phys v21, pp 1087, 1953, hereby expressly incorporated by reference.

In addition, it should be noted that the preferred methods of the invention result in a rank ordered list of sequences; that is, the sequences are ranked on the basis of some objective criteria. However, as outlined herein, it is possible to create a set of non-ordered sequences, for example by generating a probability table directly (for example using SCMF analysis or sequence alignment techniques) that lists sequences without ranking them. The sampling techniques outlined herein can be used in either situation.

In a preferred embodiment, Boltzman sampling is done. As will be appreciated by those in the art, the temperature criteria for Boltzman sampling can be altered to allow broad searches at high temperature and narrow searches close to local optima at low temperatures (see e.g., Metropolis et al., J. Chem. Phys. 21:1087, 1953).

In a preferred embodiment, the sampling technique utilizes genetic algorithms, e.g., such as those described by Holland (Adaptation in Natural and Artifical Systems, 1975, Ann Arbor, U. Michigan Press). Genetic algorithm analysis generally takes generated sequences and recombines them computationally, similar to a nucleic acid recombination event, in a manner similar to “gene shuffling”. Thus the “jumps” of genetic algorithm analysis generally are multiple position jumps. In addition, as outlined below, correlated multiple jumps may also be done. Such jumps can occur with different crossover positions and more than one recombination at a time, and can involve recombination of two or more sequences. Furthermore, deletions or insertions (random or biased) can be done. In addition, as outlined below, genetic algorithm analysis may also be used after the secondary library has been generated.

In a preferred embodiment, the sampling technique utilizes simulated annealing, e.g., such as described by Kirkpatrick et al. (Science, 220:671-680, 1983). Simulated annealing alters the cutoff for accepting good or bad jumps by altering the temperature. That is, the stringency of the cutoff is altered by altering the temperature. This allows broad searches at high temperature to new areas of sequence space, altering with narrow searches at low temperature to explore regions in detail.

In addition, the libraries may also be subsequently mutated using known techniques (exposure to mutagens, error-prone PCR, error-prone transcription, combinatorial splicing (e.g. cre-lox recombination). In this way libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

The candidate proteins may vary in size. In the case of cDNA or genomic libraries, the proteins may range from 20 or 30 amino acids to thousands, with from about 50 to 1000 being preferred and from 100 to 500 being especially preferred. When the candidate proteins are peptides, the peptides are from about 3 to about 50 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In addition, a variety of other computational methods can be used to generate the candidate protein libraries. These methods are described in U.S. Ser. No. 09/782,004, incorporated herein by reference in its entirety.

In a preferred embodiment, the nucleic acid encoding the candidate protein can also encode fusion partners. By “fusion partner” or “functional group” herein is meant a sequence that is associated with the candidate protein, that confers upon all members of the library in that class a common function or ability. Fusion partners can be heterologous (i.e. not native to the host cell), or synthetic (not native to any cell). Suitable fusion partners include, but are not limited to: a) presentation structures, as defined below, which provide the candidate proteins in a conformationally restricted or stable form, including hetero- or homodimerization or multimerization sequences; b)membrane anchoring sequences as defined below, which allow the candidate protein to be incorporated into membranes; c) membrane orientation sequences, such as CXXA-COOH, which confer a specific orientation on the protein in relation to the viral particle (Zhang and Casey, (1996) Ann. Rev. Biochem., 65:241-269); d) rescue sequences as defined below, which allow the purification or isolation of the virus particles encoding a candidate protein of interest; e) stability sequences, which confer stability or protection from degradation to the candidate protein or the nucleic acid encoding it, for example resistance to proteolytic degradation; f linker sequences; g) any number of heterologous proteins, particularly for labeling purposes as described herein; or any combination of a), b), c), d), e), f) and g), as well as linker sequences as needed.

In a preferred embodiment, the fusion partner is a presentation structure. By “presentation structure” or grammatical equivalents herein is meant a sequence, which, when fused to candidate proteins, causes the candidate proteins to assume a conformationally restricted form. This is particularly useful when the candidate proteins are random, biased random or pseudorandom peptides. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems.

Thus, synthetic presentation structures, i.e. artificial polypeptides, are capable of presenting a randomized peptide as a conformationally-restricted domain. Generally such presentation structures comprise a first portion joined to the N-terminal end of the randomized peptide, and a second portion joined to the C-terminal end of the peptide; that is, the peptide is inserted into the presentation structure, although variations may be made, as outlined below. To increase the functional isolation of the randomized expression product, the presentation structures are selected or designed to have minimal biologically activity when expressed in the target cell.

Preferred presentation structures maximize accessibility to the peptide by presenting it on an exterior loop. Accordingly, suitable presentation structures include, but are not limited to, minibody structures, dimerization sequences, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

In a preferred embodiment, the presentation structure is a coiled-coil structure, allowing the presentation of the randomized peptide on an exterior loop. See, for example, Myszka et al., Biochem. 33:2362-2373 (1994), hereby incorporated by reference). Using this system investigators have isolated peptides capable of high affinity interaction with the appropriate target. In general, coiled-coil structures allow for between 6 to 20 randomized positions.

A preferred coiled-coil presentation structure is as follows: MGCAALESEVSALESVASLESEVAALGRGDMPLAAVKSKLSAVKSKLASVKSKLAACGPP. The underlined regions represent a coiled-coil leucine zipper region defined previously (see Martin et al., EMBO J. 13(22):5303-5309 (1994), incorporated by reference). The bolded GRGDMP region represents the loop structure and when appropriately replaced with randomized peptides (i.e.candidate proteins, generally depicted herein as (X)ₙ, where X is an amino acid residue and n is an integer of at least 5 or 6) can be of variable length. The replacement of the bolded region is facilitated by encoding restriction endonuclease sites in the underlined regions, which allows the direct incorporation of randomized oligonucleotides at these positions. For example, a preferred embodiment generates a XhoI site at the double underlined LE site and a HindIII site at the double-underlined KL site.

In a preferred embodiment, the presentation structure is a minibody structure. A “minibody” is essentially composed of a minimal antibody complementarity region. The minibody presentation structure generally provides two randomizing regions that in the folded protein are presented along a single face of the tertiary structure. See for example Bianchi et al., J. Mol. Biol. 236(2):649-59 (1994), and references cited therein, all of which are incorporated by reference). Investigators have shown this minimal domain is stable in solution and have used phage selection systems in combinatorial libraries to select minibodies with peptide regions exhibiting high affinity, Kd=10⁻⁷, for the pro-inflammatory cytokine IL-6.

A preferred minibody presentation structure is as follows: MGRNSQATSGFTFSHFYMEWVRGGEYIAASRHKHNKYTTEYSASVKGRYIVSRDTSQSILYLQKKKG PP. The bold, underline regions are the regions which may be randomized. The italized phenylalanine must be invariant in the first randomizing region. The entire peptide is cloned in a three-oligonucleotide variation of the coiled-coil embodiment, thus allowing two different randomizing regions to be incorporated simultaneously. This embodiment utilizes non-palindromic BstXl sites on the termini.

In a preferred embodiment, the presentation structure is a sequence that contains generally two cysteine residues, such that a disulfide bond may be formed, resulting in a conformationally constrained sequence. This embodiment is particularly preferred when secretory targeting sequences are used. As will be appreciated by those in the art, any number of random sequences, with or without spacer or linking sequences, may be flanked with cysteine residues. In other embodiments, effective presentation structures may be generated by the random regions themselves. For example, the random regions may be “doped” with cysteine residues which, under the appropriate redox conditions, may result in highly crosslinked structured conformations, similar to a presentation structure. Similarly, the randomization regions may be controlled to contain a certain number of residues to confer β-sheet or α-helical structures.

In one embodiment, the presentation structure is a dimerization or multimerization sequence. A dimerization sequence allows the non-covalent association of one candidate protein to another candidate protein, including peptides, with sufficient affinity to remain associated under normal physiological conditions. This effectively allows small libraries of candidate protein (for example, 10⁴) to become large libraries if two proteins per cell are generated which then dimerize, to form an effective library of 10⁸ (10⁴×10⁴). It also allows the formation of longer proteins, if needed, or more structurally complex molecules. The dimers may be homo- or heterodimers.

Dimerization sequences may be a single sequence that self-aggregates, or two sequences. That is, nucleic acids encoding both a first candidate protein with dimerization sequence 1, and a second candidate protein with dimerization sequence 2, such that upon introduction into a cell and expression of the nucleic acid, dimerization sequence 1 associates with dimerization sequence 2 to form a new structure.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or even using the present methods.

In a preferred embodiment, the fusion partner is a membrane anchoring signal sequence. This is particularly useful since many parasites and pathogens bind to the membrane, in addition to the fact that many intracellular events originate at the plasma membrane. Thus, membrane-bound peptide libraries are useful for both the identification of important elements in these processes as well as for the discovery of effective inhibitors. In addition, many drugs interact with membrane associated proteins. The invention provides methods for presenting the candidate proteins extracellularly or in the cytoplasmic space. For extracellular presentation, a membrane anchoring region is provided at the carboxyl terminus of the candidate protein. In some embodiments, the membrane anchoring sequence is attached to either the N-terminus of the candidate protein or inserted in the middle of the candidate protein coding region. The candidate protein region is expressed on the cell surface and presented to the extracellular space, such that it can bind to other surface molecules (affecting their function) or molecules present in the extracellular medium. The binding of such molecules could confer function on the cells expressing a peptide that binds the molecule. The cytoplasmic region could be neutral or could contain a domain that, when the extracellular candidate protein region is bound, confers a function on the cells (activation of a kinase, phosphatase, binding of other cellular components to effect function). Similarly, the candidate protein-containing region could be contained within a cytoplasmic region, and the transmembrane region and extracellular region remain constant or have a defined function.

Membrane-anchoring sequences are well known in the art and are based on the genetic geometry of mammalian transmembrane molecules. Peptides are inserted into the membrane based on a signal sequence (designated herein as ss TM) and require a hydrophobic transmembrane domain (herein TM). The transmembrane proteins are inserted into the membrane such that the regions encoded 5′ of the transmembrane domain are extracellular and the sequences 3′ become intracellular. Of course, if these transmembrane domains are placed 5′ of the variable region, they will serve to anchor it as an intracellular domain, which may be desirable in some embodiments. ssTMs and TMs are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

As will be appreciated by those in the art, membrane-anchoring sequences, including both ssTM and TM, are known for a wide variety of proteins and any of these may be used. Particularly preferred membrane-anchoring sequences include, but are not limited to, those derived from CD8, ICAM-2, IL-8R, CD4 and LFA-1.

Useful sequences include sequences from: 1) class I integral membrane proteins such as IL-2 receptor beta-chain (residues 1-26 are the signal sequence, 241-265 are the transmembrane residues; see Hatakeyama et al., Science 244:551 (1989) and von Heijne et al, Eur. J. Biochem. 174:671 (1988)) and insulin receptor beta chain (residues 1-27 are the signal, 957-959 are the transmembrane domain and 960-1382 are the cytoplasmic domain; see Hatakeyama, supra, and Ebina et al., Cell 40:747 (1985)); 2) class 11 integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type Ill proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (MASPLTRFLSLNLLLLGESILGSGEAKPQAP; Nakauchi et al., PNAS USA 82:5126 (1985) and 1-21 in the case of ICAM-2 (MSSFGYRTLTVALFTLICCPG; Staunton et al., Nature (London) 339:61 (1989)). These leader sequences deliver the construct to the membrane while the hydrophobic transmembrane domains, placed 3′ of the random candidate region, serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR; Nakauchi, supra) and 224-256 from ICAM-2 (MVIIVTVVSVLLSLFVTSVLLCFIFGQHLRQQR; Staunton, supra).

Alternatively, membrane anchoring sequences include the GPI anchor, which results in a covalent bond between the molecule and the lipid bilayer via a glycosyl-phosphatidylinositol bond for example in DAF (PNKGSGTTSGTTRLLSGHTCFTLTGLLGTLVTMGLLT, with the bolded serine the site of the anchor; see Homans et al., Nature 333(6170):269-72 (1988), and Moran et al., J. Biol. Chem. 266:1250 (1991)). In order to do this, the GPI sequence from Thy-1 can be cassetted 3′ of the variable region in place of a transmembrane sequence.

Similarly, myristylation sequences can serve as membrane anchoring sequences. It is known that the myristylation of c-src recruits it to the plasma membrane. This is a simple and effective method of membrane localization, given that the first 14 amino acids of the protein are solely responsible for this function: MGSSKSKPKDPSQR (see Cross et al., Mol. Cell. Biol. 4(9):1834 (1984); Spencer et al., Science 262:1019-1024 (1993), both of which are hereby incorporated by reference). This motif has already been shown to be effective in the localization of reporter genes and can be used to anchor the zeta chain of the TCR. This motif is placed 5′ of the variable region in order to localize the construct to the plasma membrane. Other modifications such as palmitoylation can be used to anchor constructs in the plasma membrane; for example, palmitoylation sequences from the G protein-coupled receptor kinase GRK6 sequence (LLQRLFSRQDCCGNCSDSEEELPTRL, with the bold cysteines being palmitolyated; Stoffel et al., J. Biol. Chem 269:27791 (1994)); from rhodopsin (KQFRNCMLTSLCCGKNPLGD; Barnstable et al., J. Mol. Neurosci. 5(3):207 (1994)); and the p21 H-ras 1 protein (LNPPDESGPGCMSCKCVLS; Capon et al., Nature 302:33 (1983)).

In a preferred embodiment, the membrane anchoring sequence may be a peptide that has the ability to interact with another, known or unknown membrane protein. The interaction between the membrane anchoring sequence and the other membrane protein results in recruitment of the candidate protein to the membrane.

In a preferred embodiment, the fusion partner is a rescue sequence (sometimes also referred to herein as “purification tags” or “retrieval properties”). A rescue sequence is a sequence which may be used to purify or isolate either the candidate protein or the virus particle encoding the candidate protein. The rescue sequence may be a peptide or a non-peptide moiety or a combination of a peptide/non-peptide moiety. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST. Rescue sequences can be utilized on the basis of a binding event, an enzymatic event, a physical property or a chemical property.

As will be appreciated by those of skill in the art, the rescue tag need not be fused to the candidate protein. The rescue tag can be fused to the viral genome, the vector, endogenous proteins of the host cell, or a product formed from a reaction mediated by the candidate protein.

Alternatively, the rescue sequence may be a unique oligonucleotide sequence which serves as a probe target site to allow the quick and easy isolation of the construct, via PCR, related techniques, or hybridization.

In a preferred embodiment, the fusion partner is a stability sequence to confer stability to the candidate protein or the nucleic acid encoding it. Thus, for example, peptides may be stabilized by the incorporation of glycines after the initiation methionine (MG or MGG), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate protein structure. Thus, preferred stability sequences are as follows: MG(X)ₙGGPP, where X is any amino acid and n is an integer of at least four.

In a preferred embodiment, the fusion partner is a heterologous protein. Any number of different proteins may be added for a variety of reasons, including for labeling purposes as outlined below. Particularly suitable heterologous proteins for fusing with the candidate proteins include reporter proteins such as autofluorescent proteins. Preferred fluorescent molecules include but are not limited to green fluorescent protein (GFP; from Aquorea and Renilla species), blue fluorescent protein (BFP), yellow fluorescent protein (YFP), red fluorescent protein (RFP), and enzymes including luciferase and β-galactosidase.

In addition, the fusion partners, including presentation structures, may be modified, randomized, and/or matured to alter the presentation orientation of the randomized expression product. For example, determinants at the base of the loop may be modified to slightly modify the internal loop peptide tertiary structure, which maintaining the randomized amino acid sequence.

In a preferred embodiment, combinations of fusion partners are used. Thus, for example, any number of combinations of presentation structures, membrane anchoring sequences, rescue sequences, and stability sequences may be used, with or without linker sequences. Similarly, the fusion partners may be associated with any component of the envelope virus described herein: they may be directly fused, or be separate from these components and contained within the viral particle.

In a preferred embodiment, linkers may be used to allow functionality or flexibility. For example, linkers known to confer flexibility include glycine-serine polymers, glycine-alanine polymers, alanine-serine polymers and other flexible linkers as will be appreciated by those in the art. In addition, cleavable linkers may also be used as described in U.S. Ser. No. 09/792,629, incorporated herein in its entirety.

Thus, in a preferred embodiment, the nucleic acids of the invention comprise (1) a nucleic acid sequence encoding a candidate peptide; (2) a nucleic acid sequence comprising all or a portion of an envelope virus genome; and, (3) a fusion partner. These nucleic acids are preferably incorporated into an expression vector derived from an envelope virus, thus providing libraries of envelope virus particles. Expression vectors derived from envelope viruses are available commercially, i.e., Clontech, or may be constructed as described by Strehlow, D. (Proc. Natl. Acad. Sci. USA, 97:4209-4214, incorporated herein in their entirety.

In a preferred embodiment, the nucleic acid sequence encoding the candidate protein and/or membrane anchoring sequences is not directly fused to a viral nucleic acid sequence encoding a viral envelope protein. That is, while traditional retroviral display technologies rely on the fusion of a candidate protein to either the N- or C-terminus, an internal site, or within a few residue of the N- or C-terminals of viral proteins such as glycoproteins, the present invention need not rely on this. Preferably, the insertion of the nucleic acid sequence encoding the candidate protein does not disrupt any viral proteins necessary for viral maturation and budding. N-terminal, C-terminal, or internal fusions to a portion of the viral genome, preferably excluding sequences encoding viral glycoproteins, may be made. In addition, the sequence encoding the candidate protein may be linked to either an inducible or constitutive promoter.

In a preferred embodiment, the nucleic acid sequences encoding the candidate proteins are either present as single copies or in multiple copies. For example, multiple copies of the nucleic acids encoding the candidate proteins can be obtained by creating multiple tandem reading frames. By “multiple tandem reading frame” herein is meant a polycistronic configuration wherein expression produces more than one protein molecule. Multiple tandem reading frames may be created using internal ribosomal entry sites (IRES). The polycistronic coding fragment also be generated using DNA recombination systems known in the art. For example, a Lox-Cre site specific recombination system may be used.

In a preferred embodiment, the nucleic acid sequences encoding the candidate proteins are part of a retroviral particle which infects a host cell. Generally, infection of the cells is straightforward with the application of the infection-enhancing reagent polybrene, which is a polycation that facilitates viral binding to the target cell. Infection can be optimized such that each cell generally expresses a single construct, using the ratio of virus particles to number of cells. Infection follows a Poisson distribution.

In a preferred embodiment, the nucleic sequences encoding candidate proteins are introduced into the cells using retroviral vectors. The use of recombinant retroviruses was pioneered by Richard Mulligan and David Baltimore with the Psi-2 lines and analogous retrovirus packaging systems, based on NIH 3T3 cells (see Mann et al., Cell 33:153-159 (1993), hereby incorporated by reference). Such helper-defective packaging lines are capable of producing all the necessary trans proteins—gag, pol, and env-that are required for packaging, processing, reverse transcription, and integration of recombinant genomes. Those RNA molecules that have in cis the ψ packaging signal are packaged into maturing virions.

Particularly well suited retroviral transfection systems and vectors are available through Clontech, including, but not limited to: the pantropic retroviral expression system; retro-X-system; MSCV retroviral expression system; LRCX retroviral vector set; pSIR retroviral vector; pLEGFP-N1 retroviral vector, pLAPSN retroviral vector; pLXIN retroviral vector; pLXSN retroviral vector. Other suitable retroviral transfection systems are described in Mann et al., supra: Pear et al., PNAS USA 90(18):8392-6 (1993); Kitamura et al., PNAS USA 92:9146-9150 (1995); Kinsella et al., Human Gene Therapy 7:1405-1413; Hofmann et al., PNAS USA 93:5185-5190; Choate et al., Human Gene Therapy 7:2247 (1996); and WO 94/19478; Strehlow, et al., (2000) Proc. Natl. Acad. Sci. USA, 97:42094214; Zerangue, N., et al., (2001) Proc. Natl. Acad. Sci. USA, 98:2431-2436; and Russell et al., U.S. Pat. No. 5,723,287, and references cited therein, all of which are incorporated by reference.

The nucleic acid molecule and any of these expression vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Generally, the vectors also contain a number of other elements, including for example, the required regulatory sequences (e.g. translation, transcription, promoters, etc), fusion partners, restriction endonuclease (cloning and subcloning) sites, stop codons (preferably in all three frames), regions of complementarity for second strand priming (preferably at the end of the stop codon region as minor deletions or insertions may occur in the random region), etc.

Generally, the retroviral vectors include: inducible or constitutive promoters; selectable marker genes under the control of internal ribosome entry sites (IRES), which allows for bicistronic operons and thus greatly facilitates the selection of cells expressing peptides at uniformly high levels; and promoters driving expression of a second gene, placed in sense or anti-sense relative to the 5′ LTR. Suitable selection genes include, but are not limited to, neomycin, blastocidin, bleomycin, puromycin, and hygromycin resistance genes, as well as self-fluorescent markers such as green flourescent protein, enzymatic markers such as lacZ, and surface proteins such as CD8, etc.

Preferred vectors include: MSCV retroviral expression system; LRCX retroviral vector set; pSIR retroviral vector; pLEGFP-N1 retroviral vector, pLAPSN retroviral vector; pLXIN retroviral vector; pLXSN retroviral vector; all of which are available through Clontech.

Preferably, the nucleic acids encoding candidate proteins are first cloned into a viral shuttle vector to produce a library of plasmids. A typical shuttle vector is pLNCX (Clontech). The resultant plasmid library can then be amplified in E. coli, purified and introduced into packaging cell lines such as PT67 (Clontech).

In this manner, a library of envelope viral particles comprising nucleic acid sequences encoding candidate proteins is produced. Delivery of this library into a retroviral packaging system results in conversion to infectious virus. Suitable retroviral packaging system cell lines include, but are not limited to, the AmphoPack™-293 cell line (Clontech); the EcoPack™-293 Cell Line (Clontech); the GP2-293 Cell Line (ClontechP and the RetroPack™67 cell line (Clontech); the Bing and BOSC23 cell lines described in WO 94/19478; Soneoka et al., Nucleic Acid Res. 23(4):628 (1995); Finer et al., Blood 83:43 (1994); Pheonix packaging lines such as PhiNX-eco and PhiNX-ampho, 292T+gag-pol and retrovirus envelope; PA317; and cell lines outlined in Markowitz et al., Virology 167:400 (1988), Markowitz et al., J. Virol. 62:1120 (1988), Li et al., PNAS USA 93:11658 (1996), Kinsella et al., Human Gene Therapy 7:1405 (1996), all of which are incorporated by reference.

In a preferred embodiment, the library of envelope virus particles is used to transfect a packaging cell lines disclosed above to produce a primary viral library. By “primary viral library” herein is meant a library of envelope virus particles comprising a nucleic acid encoding a candidate protein. The production of the primary library is preferably done under conditions known in the art to reduce clone bias. The resulting primary viral library can be titred and stored, used directly to infect a target host cell line, or be used to infect another retroviral producer cell line for “expansion” of the library.

Concentration of the viral particles obtained from the primary viral library may be done as follows. Generally, retroviruses are titred by applying retrovirus-containing supernatant onto indicator cells, such as NIH3T3 cells, and then measuring the percentage of cells expressing phenotypic consequences of infection. The concentration of the virus is determined by multiplying the percentage of cells infected by the dilution factor involved, and taking into account the number of target cells available to obtain a relative titre. If the retrovirus contains a reporter gene, such as lacZ, then infection, integration, and expression of the recombinant virus is measured by histological staining for lacZ expression or by flow cytometry (FACS). In general, retroviral titres generated from even the best of the producer cells do not exceed 10⁷ per ml, unless concentration by relatively expensive or exotic apparatus. However, as it has been recently postulated that since a particle as large as a retrovirus will not move very far by brownian motion in liquid, fluid dynamics predicts that much of the virus never comes in contact with the cells to initiate the infection process. However, if cells are grown or placed on a porous filter and retrovirus is allowed to move past cells by gradual gravitometric flow, a high concentration of virus around cells can be effectively maintained at all times. Thus, up to a ten-fold higher infectivity by infecting cells on a porous membrane and allowing retrovirus supernatant to flow past them has been seen. This should allow titres of 10⁹ after concentration.

To obtain the secondary viral library host cells will preferably be infected with a multiplicity of infection (MOI) of 1/10. By “secondary viral library” herein is meant a library of envelope virus particles expressing the candidate proteins. Preferably, the candidate proteins are membrane proteins expressed on the surface of the viral envelope.

As will be appreciated by those in the art, cellular libraries comprising fusion nucleic acids encoding the viral nucleic acid and the nucleic acid sequence encoding the candidate protein are also generated using the methods described herein.

As will be appreciated by those in the art, the type of cells used in the present invention can vary widely. Any host cell capable of withstanding introduction of exogenous DNA and subsequent protein production is suitable for the present invention. The choice of the host cell will depend, in part, on the assay to be run; e.g., in vitro systems may allow the use of any number of procaryotic or eucaryotic organisms, while ex vivo systems preferably utilize animal cells, particularly mammalian cells with a special emphasis on human cells. Thus, appropriate host cells include yeast, bacteria, archaebacteria, plant, and insect and animal cells, including mammalian cells and particularly human cells. The host cells may be native cells, primary cells, including those isolated from diseased tissues or organisms, cell lines (again those originating with diseased tissues), genetically altered cells, etc. Of particular interest are Drosophila melanogaster cells, Saccharomyces cerevisiae and other yeasts, E coli, Bacillus subtilis, SF9 cells, C129 cells, 293 cells, Neurospora, BHK, CHO, COS, and HeLa cells, fibroblasts, Schwanoma cell lines, etc. See the ATCC cell line catalog, hereby expressly incorporated by reference.

Particularly preferred are mammalian cells, with mouse, rat, primate and human cells being particularly preferred, although as will be appreciated by those in the art, modifications of the system by pseudotyping allows all eukaryotic cells to be used, preferably higher eukaryotes.

In addition to the components outlined herein, including nucleic acid sequences encoding candidate proteins, linkers, fusion partners, etc., the retroviral vectors may comprise a number of additional components, including, selection genes as outlined above(particularly including growth-promoting or growth-inhibiting functions), activatible elements, recombination signals (e.g. cre and lox sites) and labels.

In a preferred embodiment, a component of the system is a labeling component. The label may be fused to one or more of the other components, for example to the nucleic acid encoding the candidate protein. The label may be fused to one or more components using chemical modification coupled with a specific amino acid side chain reaction. For example, a biotin moiety may be attached to a viral surface via a specific protein ligation step. Other methods of modification include using enzymes, such a kinases, that are specific for a specific amino acid sequence or non-amino acid moiety. In addition, as is further described below, other components of the assay systems may be labeled.

Labels can be either direct or indirect detection labels, sometimes referred to herein as “primary” and “secondary” labels. By “detection label” or “detectable label” herein is meant a moiety that allows detection. This may be a primary label or a secondary label. Accordingly, detection labels may be primary labels (i.e. directly detectable) or secondary labels (indirectly detectable).

In general, labels fall into four classes: a) isotopic labels, which may be radioactive or heavy isotopes; b) magnetic, electrical, thermal labels; c) colored or luminescent dyes or moieties; and d) binding partners. Labels can also include enzymes (horseradish peroxidase, etc.) and magnetic particles. In a preferred embodiment, the detection label is a primary label. A primary label is one that can be directly detected, such as a fluorophore.

Preferred labels include chromophores or phosphors but are preferably fluorescent dyes or moieties. Fluorophores can be either “small molecule” fluors, or proteinaceous fluors. In a preferred embodiment, particularly for labeling of target molecules, as described below, suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue™, Texas Red, Cy dyes (Cy3, Cy5, etc.), alexa dyes, phycoerythin, bodipy, and others described in the 6th Edition of the Molecular Probes Handbook by Richard P. Haugland, hereby expressly incorporated by reference.

In a preferred embodiment, for example when the label is attached to the candidate protein or is to be expressed as a component of the retroviral expression vector, proteinaceous fluores are used. Suitable autofluorescent proteins include, but are not limited to, the green fluorescent protein (GFP) from Aequorea and variants thereof; including, but not limited to, GFP, (Chalfie, et al., Science 263(5148):802-805 (1994)); enhanced GFP (EGFP; Clontech-Genbank Accession Number U55762 )), blue fluorescent protein (BFP; Quantum Biotechnologies, Inc. 1801 de Maisonneuve Blvd. West, 8th Floor, Montreal (Quebec) Canada H3H 1J9; Stauber, R. H. Biotechniques 24(3):462-471 (1998); Heim, R. and Tsien, R. Y. Curr. Biol. 6:178-182 (1996)), and enhanced yellow fluorescent protein (EYFP; Clontech Laboratories, Inc., 1020 East Meadow Circle, Palo Alto, Calif. 94303). In addition, there are recent reports of autofluorescent proteins from Renilla species. See WO 92/15673; WO 95/07463; WO 98/14605; WO 98/26277; WO 99/49019; U.S. Pat. Nos. 5,292,658; 5,418,155; 5,683,888; 5,741,668; 5,777,079; 5,804,387; 5,874,304; 5,876,995; and 5,925,558; all of which are expressly incorporated herein by reference.

In a preferred embodiment, the label protein is Aequorea green fluorescent protein or one of its variants; see Cody et al., Biochemistry 32:1212-1218 (1993); and Inouye and Tsuji, FEBS Lett. 341:277-280 (1994), both of which are expressly incorporated by reference herein.

In a preferred embodiment, a secondary detectable label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; enzymes such as horseradish peroxidase, alkaline phosphatases, lucifierases, etc; and cell surface markers, etc.

In a preferred embodiment, the secondary label is a binding partner pair. For example, the label may be a hapten or antigen, which will bind its binding partner. In a preferred embodiment, the binding partner can be attached to a solid support to allow separation of components containing the label and those that do not. For example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. In general, the smaller of the pair is attached to the system component for incorporation into the assay, although this is not required in all embodiments. Preferred binding partner pairs include, but are not limited to, biotin (or imino-biotin) and streptavidin, digeoxinin and Abs, etc.

In a preferred embodiment, the binding partner pair comprises a primary detection label (for example, attached to the assay component) and an antibody that will specifically bind to the primary detection go label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁴-10⁶M¹, with less than about 10⁵-10⁹M¹, being preferred and less 10⁷-10⁻⁹M⁻¹ being particularly preferred.

In a preferred embodiment, the secondary label is a chemically modifiable moiety. In this embodiment, labels comprising reactive functional groups are incorporated into the assay component. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference).

The end result of the above describe approaches is the expression of a candidate protein in the same environment as the nucleic acid sequence encoding it. That is, the candidate protein is expressed on the surface on the viral envelope, while the nucleic acid encoding it is located within the viral core.

Once the virus particles expressing the candidate proteins have been introduced into the host cells, the cells are lysed. Cell lysis is accomplished by any suitable technique, such as any of a variety of techniques known in the art (see, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994), hereby expressly incorporated by reference).

Following cell lysis, the supernatant is collected for virus purification as is known in the art. For example, cell debris is removed from the supernatant containing the viral particles by centrifugation at 12,000 rpm, followed by centrifugation at 25,000 rpm for 3 hours in an to obtain a virus containing pellet. The virus containing pellet is resuspended in a suitable medium and sterilized by passing through a filer with a pore diameter of 0.45 μm. If desirable, the virus particles can be further purified by centrifugation through a 20-65% sucrose gradient. Fractions can then be collected and analyzed for density or the presence of labels (see Streholow, D. et al. (2000) Proc. Natl. Acad. Sci. USA, 97:4209-4214).

Thus, the invention provides for a secondary library of envelope virus particles expressing candidate proteins. Once expressed and purified, the secondary libraries are useful in a number of applications, including in vitro, ex vivo and in vivo screening techniques. In vitro techniques include assays that are cell free, assays within cells, and assays within animals. One of ordinary skill in the art will appreciate that both in vitro and ex vivo embodiments of the present inventive method have utility in a number of fields of study. For example, the present invention has utility in diagnostic assays and can be employed for research in numerous disciplines, including, but not limited to, clinical pharmacology, functional genomics, pharamcogenomics, agricultural chemicals, environmental safety assessment, chemical sensor, nutrient biology, cosmetic research, and enzymology.

In a preferred embodiment, primary or secondary libraries of viral particles are used in in vitro screening techniques. In this embodiment, libraries of viral particles expressing candidate proteins are made and screened for binding and/or modulation of bioactivites of target molecules. One of the strengths of the present invention is to allow the identification of target molecules that bind to the candidate proteins. As is more fully outlined below, this has a wide variety of applications, including elucidating members of a signaling pathway, elucidating the binding partners of a drug or other compound of interest, etc.

Thus, libraries of envelope viruses expressing candidate proteins on the surface of the viral envelope are used in assays with target molecules. By “target molecules” or “test molecules” herein is meant molecules that are to be tested for binding to the candidate proteins expressed on the surface of the viral envelope. The test molecules in this embodiment can include a wide variety of things, including libraries of proteins, nucleic acids, lipids, carbohydrates, drugs and other small molecules, etc. In some embodiments, the target analytes comprise sets of proteins comprising different SNPs, to facilitate the identification of the role and function of different SNPs within one or more proteins.

Test molecules encompass numerous chemical classes, though typically they are organic molecules, preferably small organic compounds having a molecular weight of more than 100 and less than about 2,500 daltons. Test molecules comprise functional groups necessary for structural interaction with proteins, particularly hydrogen bonding, and typically include at least an amine, carbonyl, hydroxyl or carboxyl group, preferably at least two of the functional chemical groups. The test molecules often comprise cyclical carbon or heterocyclic structures and/or aromatic or polyaromatic structures substituted with one or more of the above functional groups. Test molecules are also found among biomolecules including peptides, saccharides, fatty acids, steroids, purines, pyrimidines, derivatives, structural analogs or combinations thereof. Particularly preferred are proteins, candidate drugs and other small molecules, and known drugs.

Test molecules are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

Suitable test molecules include organic and inorganic molecules, including biomolecules. In a preferred embodiment, the test molecule may be an environmental pollutant (including pesticides, insecticides, toxins, etc.); a chemical (including solvents, polymers, organic materials, etc.);

therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

Thus, suitable target molecules encompass a wide variety of different classes, including, but not limited to, cells, viruses, proteins (particularly including enzymes, cell-surface receptors, ion channels, and transcription factors, and proteins produced by disease-causing genes or expressed during disease states), carbohydrates, fatty acids and lipids, nucleic acids, chemical moieties such as small molecules, agricultural chemicals, drugs, ions (particularly metal ions), polymers and other biomaterials. Thus for example, binding to polymers (both naturally occurring and synthetic), or other biomaterials, may be done using the methods and compositions of the invention.

In a preferred embodiment, the test molecules are candidate proteins as defined above. In a preferred embodiment, the test molecules are naturally occurring proteins or fragments of naturally occurring proteins. Thus, for example, cellular extracts containing proteins, or random or directed digests of proteinaceous cellular extracts, may be used. In this way libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

Suitable protein test molecules include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators ( theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. famblia Y. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone, testosterone,; and (4) other proteins (including a-fetoprotein, carcinoembryonic antigen CEA.

In addition, any of the biomolecules for which antibodies are tested may be tested directly as well; that is, the virus or bacterial cells, therapeutic and abused drugs, etc., may be the test molecules.

In a preferred embodiment, the test molecules are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized test molecules.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, the test molecules are derived from cDNA libraries. The cDNA libraries can be derived from any number of different cells, particularly those outlined for host cells herein, and include cDNA libraries generated from eucaryotic and procaryotic cells, viruses, cells infected with viruses or other pathogens, genetically altered cells, etc. Preferred embodiments, as outlined below, include cDNA libraries made from different individuals, such as different patients, particularly human patients. The cDNA libraries may be complete libraries-or partial libraries. Furthermore, the library of test molecules can be derived from a single cDNA source or multiple sources; that is, cDNA from multiple cell types or multiple individuals or multiple pathogens can be combined in a screen. The cDNA library may utilize entire cDNA constructs or fractionated constructs, including random or targeted fractionation. Suitable fractionation techniques include enzymatic, chemical or mechanical fractionation.

In a preferred embodiment, the test molecules are derived from genomic libraries. As above, the genomic libraries can be derived from any number of different cells, particularly those outlined for host cells herein, and include genomic libraries generated from eucaryotic and procaryotic cells, viruses, cells infected with viruses or other pathogens, genetically altered cells, etc. Preferred embodiments, as outlined below, include genomic libraries made from different individuals, such as different patients, particularly human patients. The genomic libraries may be complete libraries or partial libraries. Furthermore, the library of test molecules can be derived from a single genomic source or multiple sources; that is, genomic DNA from multiple cell types or multiple individuals or multiple pathogens can be combined in a screen. The genomic library may utilize entire genomic constructs or fractionated constructs, including random or targeted fractionation. Suitable fractionation techniques include enzymatic, chemical or mechanical fractionation.

Suitable prokaryotic cells include, but are not limited to, bacteria such as E. coli, Bacillus species, and the extremophile bacteria such as thermophiles, etc.

Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tilapia, tuna, carp, flounder, halibut, swordfish, cod and zebrafish. Suitable bird cells include, but are not limited to, those of chickens, ducks, quail, pheasants and turkeys, and other jungle foul or game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes.

As is described herein, cell types implicated in a wide variety of disease conditions are particularly useful to identify interesting protein-protein interactions. Accordingly, suitable eukaryotic cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc. See the ATCC cell line catalog, hereby expressly incorporated by reference.

In one embodiment, the cells may be genetically engineered, that is, contain exogenous nucleic acid.

In a preferred embodiment, the test molecules are nucleic acids as defined above. As described above generally for proteins, nucleic acid test molecules may be naturally occurring nucleic acids, random nucleic acids, or “biased” random nucleic acids.

In addition, the test molecule libraries may also be subsequently mutated using known techniques (exposure to mutagens, error-prone PCR, error-prone transcription, combinatorial splicing (e.g. cre-lox recombination). In this way libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

The test molecules may vary in size. In the case of cDNA or genomic libraries, the proteins may range from 20 or 30 amino acids to thousands, with from about 50 to 1000 being preferred and from 100 to 500 being especially preferred. When the test molecules are peptides, the peptides are from about 3 to about 50 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized test molecules.

In a preferred embodiment, the test molecules are organic chemical moieties, a wide variety of which are available in the literature.

In a preferred embodiment, the test molecules are drugs, drug analogs or prodrugs. This is particularly useful to help elucidate the mechanism of drug action; for example, there are a wide variety of known drugs for which the targets and/or mechanism of action is unknown. By adding the drugs to envelope virus particles comprising candidate proteins, the proteins to which the drugs bind can be identified, and signaling and disease pathways can be constructed.

Thus, suitable target molecules encompass a wide variety of different classes, including, but not limited to, cells, viruses, proteins (particularly including secreted proteins, enzymes, cell-surface receptors, ion channels, and transcription factors, and proteins produced by disease-causing genes or expressed during disease states), non-protein attached molecules from a given cell, carbohydrates (especially cell surface moieties, such as sialyl Lewis x moiety; viral glycoproteins), fatty acids and lipids, nucleic acids, chemical moieties such as small molecules, agricultural chemicals, drugs, ions (particularly metal ions), polymers and other biomaterials. Thus for example, binding to polymers (both naturally occurring and synthetic), or other biomaterials, may be done using the methods and compositions of the invention.

In one aspect, the target is a nucleic acid sequence and the desired candidate protein has the ability to bind tot the nucleic acid sequence. The present invention may be used for the identification of DNA binding peptides and their coding sequences, as well as the target nucleic acid that are recognized and bound by the DNA binding peptides.

Current approaches used in determining protein-DNA interactions are focused on studying the individual interactions between DNA and specific protein targets. A variety of biochemical and molecular assays including DNA footprinting, nuclease protection, gel shift, and affinity chromatographic binding are employed to study protein-DNA interactions. Although these methods are useful for detecting individual DNA-protein interactions, they are not suitable for large-scale analyses of these interactions at the genomic level. Thus, there is a need in the art to perform large-scale analyses of DNA binding proteins and their interacting DNA sequences. The methods and libraries of the present invention are useful for such analyses. For example, the viral particle library encoding potential DNA binding peptides can be screened against a population of target DNA segments. The population of target DNA segments can be, for instance, random DNA, fragmented genomic DNA, degenerate sequences, or DNA sequences of various primary, secondary or tertiary structures. The specificity of the DNA binding peptide-substrate binding can be varied by changing the length of the recognition sequence of the target DNA, if desired. Binding of the potential DNA binding peptide to a member of the population of target DNA segments is detected, and further study of the particular DNA recognition sequence bound by the DNA binding peptide can be performed. To facilitate identification of viral particle-nucleic acid complexes, the population of DNA segments can be bound to, for example, beads or constructed as DNA arrays on microchips. Therefore, using the present inventive method, one of ordinary skill in the art can identify DNA binding peptides, identify the coding sequence of the DNA binding peptides, and determine what nucleic acid sequence the DNA binding peptides recognize and bind. Thus, in one embodiment, the present invention provides methods for creating a map of DNA binding sequences and DNA binding proteins according to their relative positions, to provide chromosome maps annotated with proteins and sequences.

Thus, libraries of envelope virus particles expressing candidate proteins are used in screens to assay binding to target molecules and/or to screen candidate agents for the ability to modulate the activity of the target molecule.

In general, screens are designed to first find candidate proteins that can bind to target molecules, and then these proteins are used in assays that evaluate the ability of the candidate protein to modulate the target's bioactivity. Thus, there are a number of different assays which may be run; including binding assays and activity assays. As will be appreciated by those in the art, these assays may be run in a variety of configurations, including both solution-based assays and utilizing support-based systems.

In a preferred embodiment, the assays comprise combining the viral libraries of the invention and a target molecule, and determining the binding of the candidate protein expressed on the surface of the viral envelope to the target molecule. Preferably, libraries of envelope virus particles (e.g. comprising a library of different candidate proteins) are contacted with either a single type of target molecule, a plurality of target molecules, or one or more libraries of target molecules.

Generally, in a preferred embodiment of the methods herein, one of the components of the invention, either the envelope virus particle expressing the candidate protein or the target molecule, is non-diffusably bound to an insoluble support having isolated sample receiving areas (e.g. a microtiter plate, an array, etc.). The insoluble support may be made of any composition to which the assay component can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. Alternatively, bead-based assays may be used, particularly with use with fluorescence activated cell sorting (FACS). The particular manner of binding the assay component is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the use of labeled components (e.g. the assay component is biotinylated and the surface comprises strepavidin, etc. the synthesis of the target on the surface, etc. Following binding of the candidate protein or target molecule, excess unbound material is removed by washing. The sample receiving areas may then be blocked through incubation with bovine serum albumin (BSA), casein or other innocuous protein or other moiety.

In a preferred embodiment, the target molecule is bound to the support, and an envelope virus particle expressing a candidate protein is added to the assay. Alternatively, the envelope virus particle expressing a candidate protein is bound to the support and the target molecule is added. Novel binding agents include specific antibodies, non-natural binding agents identified in screens of chemical libraries, peptide analogs, etc. Of particular interest are screening assays for agents that have a low toxicity for human cells. Determination of the binding of the target and the candidate protein is done using a wide variety of assays, including, but not limited to labeled in vitro protein-protein binding assays, electrophoretic mobility shift assays, immunoassays for protein binding, the detection of labels, functional assays (phosphorylation assays, etc.) and the like.

The determination of the binding of the candidate protein to the target molecule may be done in a number of ways. In a preferred embodiment, one of the components, preferably the soluble one, is labeled, and binding determined directly by detection of the label. For example, this may be done by attaching the envelope virus particle expressing a candidate protein to a solid support, adding a labeled target molecule (for example a target molecule comprising a fluorescent label), washing off excess reagent, and determining whether the label is present on the solid support. This system may also be run in reverse, with the target (or a library of targets) being bound to the support and envelope viruses expressing candidate proteins, preferably comprising a primary or secondary label, added. For example, envelope virus particles expressing a candidate protein comprising fusions with GFP or a variant may be particularly useful. Various blocking and washing steps may be utilized as is known in the art.

As will be appreciated by those in the art, it is also possible to contact the envelope viruses expressing the candidate proteins and the targets prior to immobilization on a support.

In a preferred embodiment, the solid support is in an array format; that is, a biochip is used which comprises one or more libraries of either targets or libraries of envelope virus particles expressing candidate proteins attached to the array. The biochips comprise a substrate. By “substrate” or “solid support” or other grammatical equivalents herein is meant any material appropriate for the attachment of capture probes and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, ceramics, and a variety of other polymers. In a preferred embodiment, the substrates allow optical detection and do not themselves appreciably fluoresce.

This can find particular use in assays for nucleic acid binding proteins, as nucleic acid biochips are well known in the art. In this embodiment, the nucleic acid targets are on the array and envelope virus particles expressing candidate proteins are added. Similarly, protein biochips of libraries of target proteins can be used, with labeled envelope virus particles expressing candidate proteins added.

Alternatively, the libraries of virus particles can be attached to the chip, either through the nucleic acid or through the protein components of the system. See also U.S. Ser. No. 09/792,630, filed Feb. 22, 25, 2001, no serial number received yet, hereby expressly incorporated by reference.

This may also be done using bead based systems; for example, for the detection of nucleic acid binding proteins, standard “split and mix” techniques, or any standard oligonucleotide synthesis schemes, can be run using beads or other solid supports, such that libraries of sequences are made. The addition of envelope virus libraries then allows for the detection of candidate proteins that bind to specific sequences.

In some embodiments, only one of the components is labeled; alternatively, more than one component may be labeled with different labels.

In a preferred embodiment, the binding of the candidate protein is determined through the use of competitive binding assays. In this embodiment, the competitor is a binding moiety known to bind to the target molecule such as an antibody, peptide, binding partner, ligand, etc. Under certain circumstances, there may be competitive binding as between the target and the binding moiety, with the binding moiety displacing the target.

Thus, a preferred utility of the invention is to determine the components to which a drug will bind. That is, there are many drugs for which the targets upon which they act are unknown, or only partially known.

By starting with a drug, and envelope virus particles comprising a library of cDNA expression products from the cell type on which the drug acts, the elucidation of the proteins to which the drug binds may be elucidated. By identifying other proteins or targets in a signaling pathway, these newly identified proteins can be used in additional drug screens, as a tool for counterscreens, or to profile chemically induced events. Furthermore, it is possible to run toxicity studies using this same method; by identifying proteins to which certain drugs undesirably bind, this information can be used to design drug derivatives without these undesirable side effects. Additionally, drug candidates can be run in these types of screens to look for any or all types of interactions, including undesirable binding reactions. Similarly, it is possible to run libraries of drug derivatives as the targets, to provide a two-dimensional analysis as well.

Positive controls and negative controls may be used in the assays Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound. Similarly, ELISA techniques are generally preferred.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, co-factors such as cAMP, ATP, etc., may be used. The mixture of components may be added in any order that provides for the requisite binding. Screening for agents that modulate the activity of the target molecule may also be done. As will be appreciated by those in the art, the actual screen will depend on the identity of the target molecule. In a preferred embodiment, methods for screening for a candidate protein capable of modulating the activity of the target molecule comprise the steps of adding an envelope virus particle expressing a candidate protein to a sample of the target, as above, and determining an alteration in the biological activity of the target. “Modulation” or “alteration” in this context includes an increase in activity, a decrease in activity, or a change in the type or kind of activity present. Thus, in this embodiment, the candidate protein should both bind to the target (although this may not be necessary), and alter its biological or biochemical activity as defined herein. The methods include both in vitro screening methods, as are generally outlined above, and ex vivo screening of cells for alterations in the presence, distribution, activity or amount of the target.

Thus, in this embodiment, the methods comprise combining a target molecule and preferably a library of envelope viruses expressing candidate proteins and evaluating the effect on the target molecule's bioactivity. This will be done in a wide variety of ways, as will be appreciated by those in the art.

In these in vitro systems, e.g. cell-free systems, in either embodiment, e.g. in vitro binding or activity assays, once a “hit” is found, the envelope virus particle expressing the candidate protein is retrieved to allow identification of the candidate protein. Retrieval of the viral particle can be done in a wide variety of ways, as will be appreciated by those in the art and will also depend on the type and configuration of the system being used.

In a preferred embodiment, as outlined herein, a rescue tag or“retrieval property” is used. As outlined above, a “retrieval property” is a property that enables isolation of the viral particle when-bound to the target. For example, the target can be constructed such that it is associated with biotin, which enables isolation of the target-bound viral particle complexes using an affinity column coated with streptavidin. Alternatively, the target can be attached to magnetic beads, which can be collected and separated from non-binding candidate proteins by altering the surrounding magnetic field. Alternatively, when the target does not comprise a rescue tag, the envelope virus particle expressing the candidate protein may comprise the rescue tag. For example, affinity tags may be incorporated into the nonviral candidate protein. Similarly, the fusion nucleic acid molecule complex can be also recovered by immunoprecipitation. Alternatively, rescue tags may comprise unique vector sequences that can be used to PCR amplify the nucleic acid encoding the candidate protein. In the latter embodiment, it may not be necessary to break the covalent attachment of the nucleic acid and the protein, if PCR sequences outside of this region (that do not span this region) are used.

The nucleic acid molecules are purified using any suitable methods, such as those methods known in the art, and are then available for further amplification, sequencing or evolution of the nucleic acid sequence encoding the desired candidate protein. Suitable amplification techniques include all forms of PCR, OLA, SDA, NASBA, TMA, Q-PR, etc. Subsequent use of the information of the “hit” is discussed below.

In a preferred embodiment, libraries of envelope viruses expressing candidate proteins are used to determine if the tropism of a given virus particle is altered. By “trophism” herein is meant a property of the virus, such as host range, infectivity, etc., is altered due the expression of the candidate protein on the surface of the viral envelope. As will be appreciated by one of skill in the art, different conditions can be used to screen for a specific tropism by testing a plurality of viral particles expressing different candidate proteins.

Screening for changes in trophism can be done in several ways.- In-a preferred embodiment, a library of viral particles expressing the candidate protein is used to infect a host cell. The infected host cells are then screened for changes in viral trophisms, such as infectivity, a switch from lysogenic to lytic state, change in the cell phenotype, such as a switch from a wildype cell to a cancerous cell. Cells exhibiting an altered response to the virus are then isolated and the virus particle recovered as described herein.

In a preferred embodiment, ex vivo screening techniques are used to screen cellular libraries in which the envelope viral genome, including the nucleic acid sequence encoding the candidate protein, is present in a proviral state. In this embodiment, libraries of virus particles are introduced into the cells to screen for candidate proteins capable of altering the phenotype of a cell. An advantage of the present inventive method is that screening of the library can be accomplished intracellularly. One of ordinary skill in the art will appreciate the advantages of screening candidate proteins within their natural environment, as opposed to lysing the cell to screen in vitro. In ex vivo screening methods, candidate proteins are displayed in their native conformation and are screened in the presence of other possibly interfering or enhancing cellular agents. Accordingly, screening intracellularly provides a more accurate picture of the actual activity of the candidate protein and, therefore, is more predictive of the activity of the peptide ex vivo. Moreover, the effect of the candidate protein on cellular physiology can be observed. Thus, the invention finds particular use in the screening of eucaryotic cells.

As will be appreciated by those in the art, the type of cells used in this embodiment can vary widely. Basically, any eucaryotic or procaryotic cells can be used, with mammalian cells being preferred, especially mouse, rat, primate and human cells.

In addition, for both in vitro and ex vivo screening methods, the process may be used reiteratively. That is, the sequence of a candidate protein is used to generate more candidate proteins. For example, the sequence of the protein may be the basis of a second round of (biased) randomization, to develop agents with increased or altered activities. Alternatively, the second round of randomization may change the affinity of the agent. Furthermore, if the candidate protein is a random peptide, it may be desirable to put the identified random region of the agent into other presentation structures, or to alter the sequence of the constant region of the presentation structure, to alter the conformation/shape of the candidate protein.

The methods of using the present inventive library can involve many rounds of screening in order to identify a nucleic acid of interest. For example, once a nucleic acid molecule is identified, the method can be repeated using a different target. Multiple libraries can be screened in parallel or sequentially and/or in combination to ensure accurate results. In addition, the method can be repeated to map pathways or metabolic processes by including an identified candidate protein as a target in subsequent rounds of screening.

In a preferred embodiment, the candidate protein is used to identify target molecules, i.e. the molecules with which the candidate protein interacts. As will be appreciated by those in the art, there may be primary target molecules, to which the protein binds or acts upon directly, and there may be secondary target molecules, which are part of the signalling pathway affected by the protein agent; these might be termed “validated targets”.

In a preferred embodiment, the candidate protein is used to pull out target molecules. For example, as outlined herein, if the target molecules are proteins, the use of epitope tags or purification sequences can allow the purification of primary target molecules via biochemical means (co-immunoprecipitation, affinity columns, etc.). Alternatively, the peptide, when expressed in bacteria and purified, can be used as a probe against a bacterial cDNA expression library made from mRNA of the target cell type. Or, peptides can be used as “bait” in either yeast or mammalian two or three hybrid systems. Such interaction cloning approaches have been very useful to isolate DNA-binding proteins and other interacting protein components. The peptide(s) can be combined with other pharmacologic activators to study the epistatic relationships of signal transduction pathways in question. It is also possible to synthetically prepare labeled peptides and use it to screen a cDNA library expressed in bacteriophage for those cDNAs which bind the peptide.

Once primary target molecules have been identified, secondary target molecules may be identified in the same manner, using the primary target as the “bait”. In this manner, signalling pathways may be elucidated. Similarly, protein agents specific for secondary target molecules may also be discovered, to allow a number of protein agents to act on a single pathway, for example for combination therapies.

In a preferred embodiment, the methods and compositions of the invention comprise a robotic system. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.

In a preferred embodiment, interchangeable pipet heads (single or multi-channel ) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some preferred embodiments, the instrumentation will include a detector, which can be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems, for cell culture growth and transformation in multi-well plates or tubes and for hazardous operations. The living cells will be grown under controlled growth conditions, with controls for temperature, humidity, and gas for time series of the live cell assays. Automated transformation of cells and automated colony pickers will facilitate rapid screening of desired cells.

Flow cytometry or capillary electrophoresis formats can be used for individual capture of magnetic and other beads, particles (including the viral particles with or without an associated target molecule), cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which can be done using any number of known heating and cooling systems, including Peltier systems.

In a preferred embodiment, the robotic apparatus includes a central processing unit which communicates with a memory and a set of input/output devices (e.g., keyboard, mouse, monitor, printer, etc.) through a bus. The general interaction between a central processing unit, a memory, input/output devices, and a bus is known in the art. Thus, a variety of different procedures, depending on the experiments to be run, are stored in the CPU memory.

The above-described methods of screening a pool of envelope virus particles for a nucleic acid encoding a desired candidate protein are merely based on the desired target property of the candidate protein. The sequence or structure of the candidate proteins does not need to be known. Moreover, the present methods provide a means of expressing membrane proteins in an appropriate environment, that is on the surface of a bilayer membrane. A significant advantage of the present invention is that no prior information about the candidate protein is needed during the screening, so long as the product of the identified coding nucleic acid sequence has biological activity, such as specific association with a targeted chemical or structural moiety. The identified nucleic acid molecule then can be used for understanding cellular processes as a result of the candidate protein's interaction with the target and, possibly, any subsequent therapeutic or toxic activity.

All references cited herein are incorporated by reference.

We claim:

1. A library of fusion nucleic acids each comprising:
a) a nucleic acid sequence comprising an envelope viral genome;
b) a nucleic acid sequence encoding a candidate protein wherein said nucleic acid is not directly fused to a viral glycoprotein gene; and
c) a nucleic acid sequence encoding a membrane anchoring sequence wherein said nucleic acid encoding said candidate protein and said membrane anchoring sequence are fused; wherein at least two of said candidate proteins are different.

2. The library according to claim 1 wherein said membrane anchoring sequence is endogenous.

3. The library according to claim 1 wherein said membrane anchoring sequence is exogenous.

4. The library according to claim 1 wherein said nucleic acid encoding said candidate protein and said membrane anchoring sequence are directly fused.

5. The library according to claim 1 wherein said nucleic acid encoding said candidate protein and said membrane anchoring sequence are indirectly fused.

6. The candidate protein of claim 1 wherein the candidate protein is a random peptide.

7. A library according to claim 1 wherein said candidate protein is a cell membrane protein.

8. A cellular library comprising:
a) a nucleic acid sequence comprising an envelope viral genome;
b) a nucleic acid sequence encoding a candidate protein wherein said nucleic acid is not directly fused to a viral glycoprotein gene; and
c) a nucleic acid sequence encoding a membrane anchoring sequence wherein said nucleic acid encoding said candidate protein and said membrane anchoring sequence are fused; wherein at least two of said candidate proteins are different.

9. The cellular library according to claim 8 wherein said cells are eucaryotic.

10. The cellular library according to claim 8 wherein said eucaryotic cells are from humans.

11. A library of envelope virus particles comprising:
a) a nucleic acid sequence comprising an envelope viral genome;
b) a nucleic acid sequence encoding a candidate protein wherein said nucleic acid is not directly fused to a viral glycoprotein gene; and
c) a nucleic acid sequence encoding a membrane anchoring sequence wherein said nucleic acid encoding said candidate protein and said membrane anchoring sequence are fused; wherein at least two of said candidate proteins are different.

12. A library according to claims 1, 8, or 11 wherein said envelope virus is a member of the family Retroviridae.

13. A library according to claims 1, 8, or 11 wherein said nucleic acid comprising said viral genome is RNA.

14. A library according to claims 1, 8, or 11 wherein said nucleic acid comprising said nucleic acid is DNA.

15. A method for generating a library of envelope viral particles comprising:
a) inserting a nucleic acid encoding a candidate protein into an envelope virus vector;
b) transfecting a packaging cell line to produce a primary viral library; and
c) transducing a host cell with said primary viral library to produce a secondary viral library.

16. A method according to claim 15 wherein multiple copies of said nucleic acid encoding a candidate protein are inserted.

17. A method according to claim 15 wherein said host cell is eucaryotic.

18. A method for expressing a candidate protein on the surface of an envelope viral particle comprising:
a) providing a library of viral vectors comprising a nucleic acid comprising:
i) first nucleic acid encoding an envelope virus genome; and,
ii) a second nucleic acid encoding a candidate protein;
b) expressing said nucleic acid under conditions whereby a library of virus particles are formed expressing said candidate membrane protein on the surface of the said virus particles, wherein at least two of the candidate membrane proteins are different.

19. A method for screening comprising:
a) providing a library of envelope virus particles comprising at least one fusion nucleic acid comprising:
i) a nucleic acid sequence comprising an envelope viral genome;
ii) a nucleic acid sequence encoding a candidate protein wherein said nucleic acid is not directly fused to a viral glycoprotein gene; and
iii) a nucleic acid sequence encoding a membrane anchoring sequence wherein said nucleic acid encoding said candidate protein and said membrane anchoring sequence are fused;
wherein at least two of said candidate proteins are different;
b) expressing said fusion nucleic acid under conditions whereby a library of viral particles are formed, wherein said viral particles comprise:
i) a nucleic acid sequence encoding a membrane anchoring sequence wherein said nucleic acid encoding said candidate protein and said membrane anchoring sequence are fused;
ii) a nucleic acid comprising an envelope virus genome;
ii) said candidate protein;
c) adding at least one test molecule to said library; and
d) determining the binding of said candidate protein.

20. A method of screening for membrane anchoring sequences comprising:
a) providing a library of envelope virus particles comprising at least one fusion nucleic acid comprising:
i) a nucleic acid sequence comprising an envelope virus genome;
ii) a nucleic acid sequence comprising a soluble protein wherein said nucleic acid is not directly fused to a viral glycoprotein gene; and
iii) a nucleic acid sequence encoding a candidate membrane anchoring sequence wherein said nucleic acid encoding said candidate protein and said membrane anchoring sequence are fused;
wherein at least two of said membrane anchoring sequences are different;
b) expressing said fusion nucleic acid under conditions whereby a library of viral particles are formed, wherein said viral particles comprise:
i) a nucleic acid sequence encoding a membrane anchoring sequence wherein said nucleic acid encoding said candidate protein and said membrane anchoring sequence are fused;
ii) a nucleic acid comprising an envelope virus genome;
ii) a fusion polypeptide encoding said soluble protein and said candidate membrane anchoring sequence; and,
c) determining if said soluble protein is anchored to the viral envelope via said candidate membrane anchoring sequence.
